(19) United States
(12) Patent Application Publication
(10) Pub. No.: US 2018/0296530 A1
(43) Pub. Date: Oct. 18, 2018

(54) METHOD FOR TREATING ATOPIC DERMATITIS

(71) Applicants: Bor-Luen Chiang, Taipei City (TW); Yung-Sen Chang, Taipei City (TW)

(72) Inventors: Bor-Luen Chiang, Taipei City (TW); Yung-Sen Chang, Taipei City (TW)

(21) Appl. No.: 16/065,151

(22) PCT Filed: Sep. 6, 2016
(86) PCT No.: PCT/CN2016/098244

§ 371 Date: Jun. 22, 2018

Related U.S. Application Data
(60) Provisional application No. 62/387,152, filed on Dec. 23, 2015.

(51) Int. Cl. A61K 31/4045; A61P 17/04

(52) U.S. Cl. CPC A61K 31/4045; A61P 17/04

(57) ABSTRACT

A method for treating atopic dermatitis in a subject in need thereof is provided. The method includes administering to the subject a composition containing a therapeutically effective amount of a melatonin associated compound and a pharmaceutically acceptable excipient.

### BACKGROUND OF THE DISCLOSURE

### 1. Field of the Disclosure

The present disclosure relates to a method for treating atopic dermatitis, and more particularly relates to a method for treating atopic dermatitis in a subject with atopic dermatitis by a melatonin associated compound.

### 2. Description of Related Art

Atopic dermatitis (AD) is a common chronic, relapsing, pruritic inflammatory skin disease affecting 15% to 30% of all children and 2% to 10% of all adults. [1] Disturbed sleep is reported in 47% to 60% of children with AD and is a major factor leading to an impaired quality of life. [2-4]

At present, limited studies are available to guide effective clinical management for sleep disturbances in AD. Some investigators have studied whether treatment targeted at skin inflammation in patients with AD also improves their sleep, [5, 6] but the results have been inconsistent, and most of these studies used a subjective visual analog scale rather than objective measures to evaluate sleep.

First-generation antihistamines are traditionally used for sleep problems in patients with AD because they can antagonize the inflammatory effects of histamine released from mast cells and basophils and can cross the blood-brain barrier and thereby affect histamine's role in maintaining central nervous system arousal, which results in a sedating effect.[7] However, tolerance often occurs after 4 to 7 days of treatment and the sedating effect vanishes, limiting its usefulness. [8, 9] Anticholinergic adverse effects, such as blurred vision and dry mouth, are also major concerns.

Benzodiazepines have sedating and anxiolytic effects, but they carry the risks for tolerance to sedating effects, rebound worsening of sleep problems at discontinuation, and addiction. Negative adverse effects, including muscle relaxation and memory problems, also make them less favorable for use in children. [7]

There is therefore an ongoing need for a new, potent and therapeutically effective method for improving the skin inflammation and sleep disturbances with AD.

### SUMMARY

Exemplary embodiments of the general inventive concept provide a method for treating atopic dermatitis in a subject in need thereof comprising administering to the subject a composition comprising a therapeutically effective amount of a melatonin associated compound and a pharmaceutically acceptable excipient.

According to the embodiments of the present disclosure, the melatonin associated compound is at least one selected from the group consisting of melatonin (MLT), melatonin receptor agonists, Ramelton, Agomelatine, Tasimelteon, TIK-301, halogenated melatonin, 2-iodomelatonin, 2-bromomelatonin, 2-phenylmelatonin, 6-chloromelatonin, and the following compounds represented by Formulae 1-28:

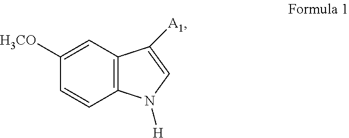

wherein A₁ is CH₂CH₂NHCOCH₃;

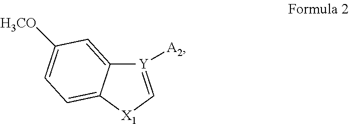

wherein A₂ is CH₂CH₂NHCOCH₃, and X₁ is S, O or CH₂ when Y is CH, or X₁ is NH when Y is N;

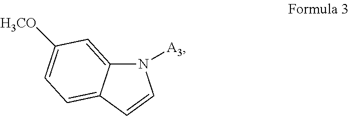

wherein A₃ is CH₂CH₂NHCOCH₃;

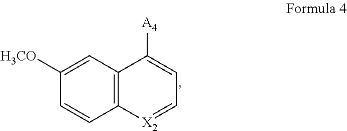

wherein A₄ is CH₂CH₂NHCOCH₃, and X₂ is CH or N;

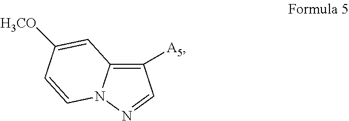

wherein A₅ is CH₂CH₂NHCOCH₃;

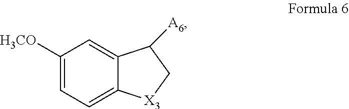

wherein A₆ is CH₂CH₂NHCOCH₃, and X₃ is NH or CH₂;

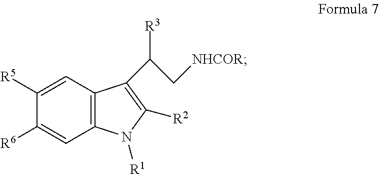

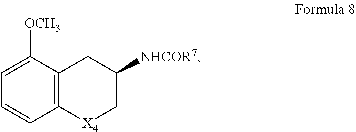

wherein X₄ is CH₂ when R⁷ is methyl, or X₄ is O when R⁷ is n-propyl;

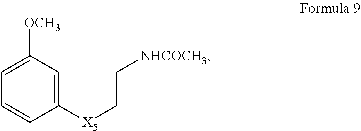

wherein X₅ is CH₂ or NCH₃;

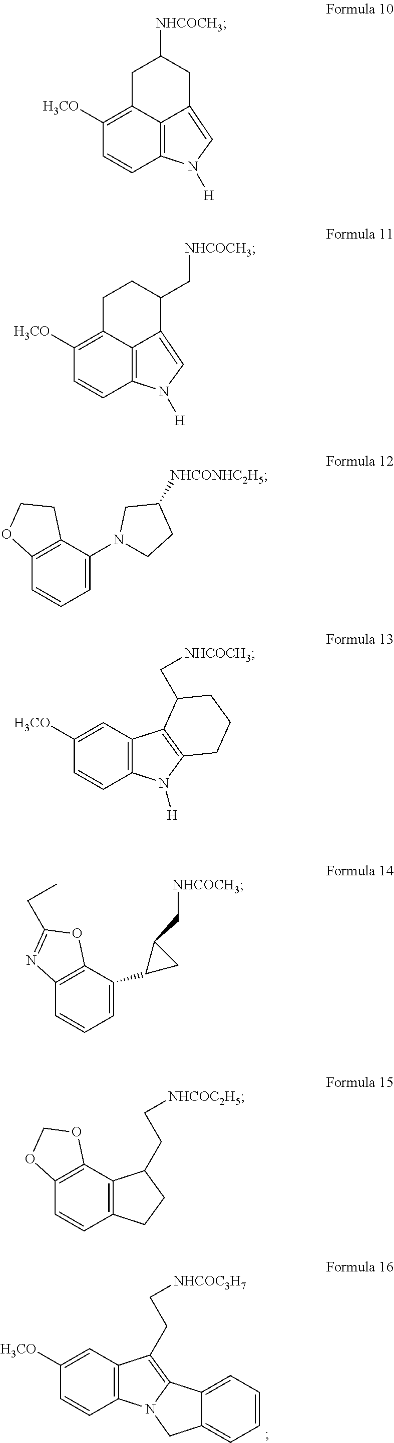

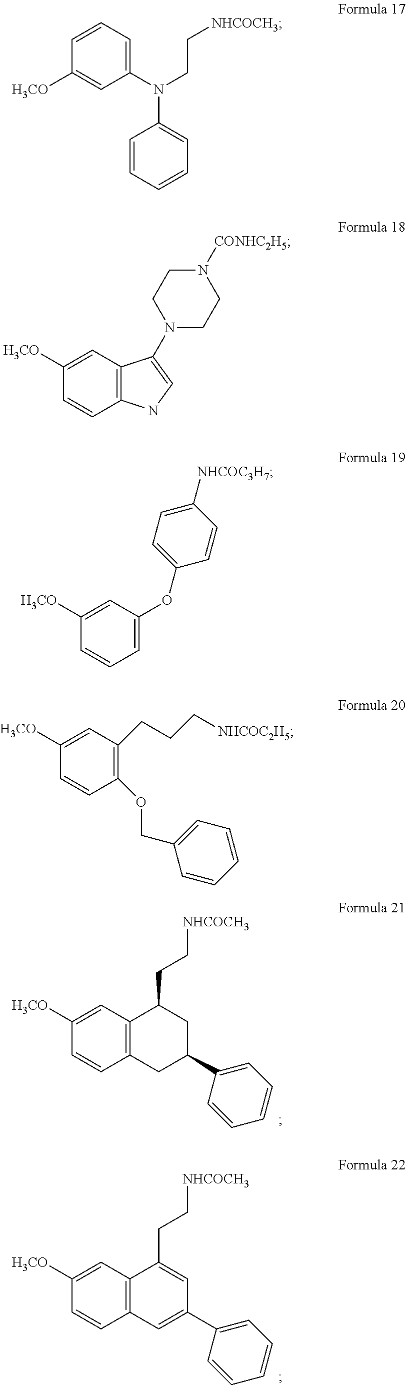

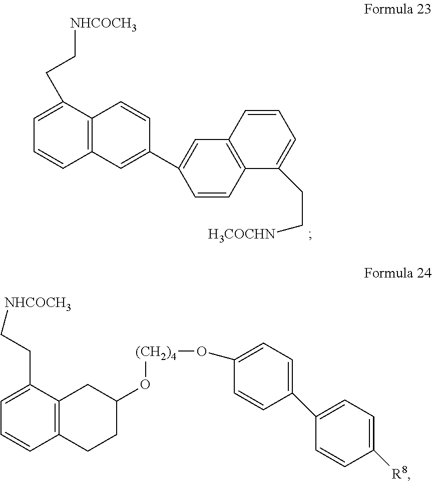

wherein R⁸ is COOH or CH₂OH;

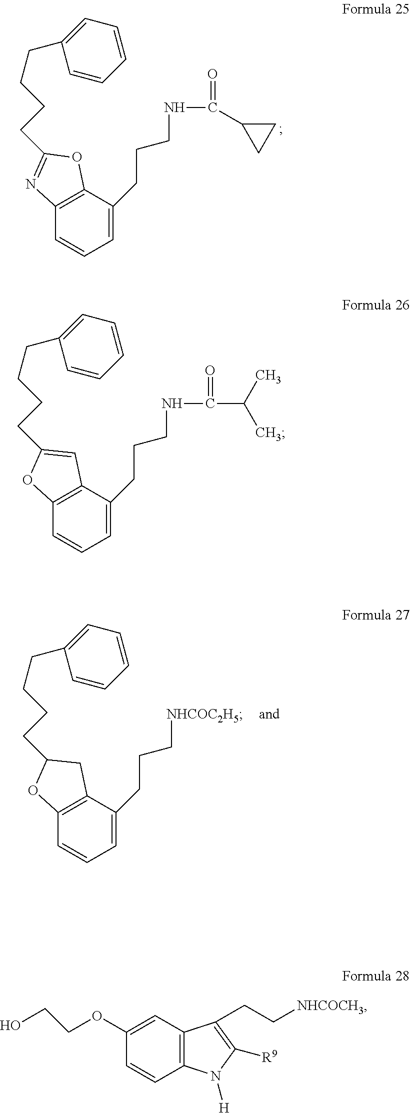

wherein R⁹ is H or Br.

According to the embodiments of the present disclosure, the therapeutically effective amount of a melatonin associated compound decreases the sleep-onset latency of the subject to ameliorate or treat sleep disturbance.

According to the embodiments of the present disclosure, the melatonin associated compound in the composition is in an amount of from 0.001% to 30% by weight. In another embodiment, the melatonin associated compound in the composition is in an amount of from 0.1% to 10% by weight.

According to the embodiments of the present disclosure, the melatonin associated compound is administered to the subject in an amount ranging from 0.0001 mg/day to 50 mg/day. In another embodiment, the melatonin associated compound is administered to the subject in an amount ranging from 0.01 mg/day to 25 mg/day. In yet another embodiment, the melatonin associated compound is administered to the subject in an amount of about 3 mg/day.

According to the embodiments of the present disclosure, the composition is administered to the subject 1 to 4 times daily. In another embodiment, the composition is administered to the subject 2 times daily.

According to the embodiments of the present disclosure, the composition is orally, intravenously, intraperitoneally, intramuscularly, externally or transdermally administered to the subject.

According to the embodiments of the present disclosure, the composition is administered to the subject in a period sufficient to ameliorate atopic dermatitis. According to the embodiment of the present disclosure, the period is in a range of from 3 days to 2 years. In another embodiment, the period is in a range of from 2 weeks to 12 months. In yet another embodiment, the period is in a range of from 2 to 4 weeks.

According to the embodiments of the present disclosure, the method further comprises administering an additional therapeutic agent for treating atopic dermatitis at the same time of administering the melatonin associated compound.

According to the embodiments of the present disclosure, the method further comprises administering an additional therapeutic agent for treating atopic dermatitis separately from administering the melatonin associated compound.

In one embodiment, the additional therapeutic agent is at least one selected from a steroid, an anti-histamine agent, cetirizine, loratadine, chlorpheniramine, and a natural or synthetic immune inhibitor.

According to the embodiments of the present disclosure, the method is in combination with an additional therapy for preventing or treating atopic dermatitis. In one embodiment, the additional therapy is at least one chosen from phototherapy, skin care, protection from allergen exposure, or administration of a skin moisturizer, an adjuvant, an antioxidant, a vitamin, or a mineral.

### BRIEF DESCRIPTION OF DRAWINGS

The present disclosure can be more fully understood by reading the following detailed description of the embodiments, with reference made to the accompanying drawings, wherein:

FIG. 1 is a diagram of the clinical trial design of embodiments of the present disclosure;

FIGS. 2A to 2D show severity of AD and sleep-onset latency before and after treatment presented by SCORAD index (FIG. 2A), treatment sequence (FIG. 2B), objective SCORAD index (FIG. 2C), and sleep-onset latency (FIG. 2D);

FIGS. 3A and 3B respectively show sleep efficiency (FIG. 3A) and fragmentation index (FIG. 3B) before and after treatment;

FIG. 4 is a diagram of the protocol for the treatment in the AD mouse model;

FIG. 5 shows gross findings of AD mouse treated with or without melatonin;

FIG. 6 shows H&E stained skin tissues of AD mouse treated with or without melatonin;

FIG. 7 shows pathology scores of the skin of AD mouse treated with or without melatonin (*p<0.01 compared with Control; #p<0.05 compared with DNCB);

FIG. 8 shows mast cell infiltration in the skin of AD mouse treated with or without melatonin evaluated with toluidine blue stain;

FIGS. 9A and 9B show immunohistochemistry staining for anti-CD3 positive cells in the skin of AD mouse treated with or without melatonin and its bar chart (*p<0.01 compared with Control; #p<0.05 compared with DNCB);

FIG. 10 shows serum total IgE level in AD mouse treated with or without melatonin (*p<0.01 compared with Control; #p<0.05 compared with DNCB);

FIGS. 11A and 11B show mRNA expression of IL-4 and IL-17 in the skin of AD mouse treated with or without melatonin;

FIG. 12 is a diagram of the protocol for the treatment in the AD mouse model;

FIG. 13 shows gross findings of AD mouse treated with or without melatonin;

FIG. 14 is a bar chart of ear thickness of AD mouse treated with or without melatonin (*p<0.01 compared with Control; #p<0.01 compared with DNCB);

FIG. 15 shows serum total IgE level in AD mouse treated with or without melatonin.

### DETAILED DESCRIPTION

Reference will now be made in detail to the embodiments of the present general inventive concept, examples of which are illustrated in the accompanying drawings. The embodiments are described below in order to explain the present general inventive concept while referring to the figures.

All terms including descriptive or technical terms which are used herein should be construed as having meanings that are obvious to one of ordinary skill in the art. However, the terms may have different meanings according to an intention of one of ordinary skill in the art, case precedents, or the appearance of new technologies. Also, some terms may be arbitrarily selected by the applicant, and in this case, the meaning of the selected terms will be described in detail in the detailed description of the present disclosure. Thus, the terms used herein have to be defined based on the meaning of the terms together with the description throughout the specification.

Also, when a part “includes” or “comprises” a component or a step, unless there is a particular description contrary thereto, the part can further include other components or other steps, not excluding the others. In the following description, terms such as “a” and “one” indicate a unit to process at least one function or operation, wherein the unit and the block may be embodied as hardware or software or embodied by combining hardware and software.

It should be noted that, as used in this specification, the singular forms “a,” “an,” and “the” include plural referents unless expressly and unequivocally limited to one referent. The term “or” is used interchangeably with the term “and/or” unless the context clearly indicates otherwise.

The present inventive concept provides a method for treating atopic dermatitis (AD) in a subject comprising administering to the subject a composition comprising a therapeutically effective amount of a melatonin associated compound and a pharmaceutically acceptable excipient, wherein the subject can be a subject with atopic dermatitis.

According to the present inventive concept, the melatonin associated compound may be at least one selected from the group consisting of melatonin (MLT), melatonin receptor agonists, Ramelton, Agomelatine, Tasimelteon, TIK-301, halogenated melatonin, 2-iodomelatonin, 2-bromomelatonin, 2-phenylmelatonin, 6-chloromelatonin, and the following compounds represented by Formulae 1-28:

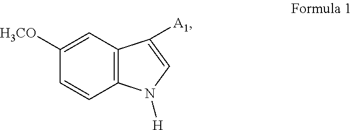

wherein A₁ is CH₂CH₂NHCOCH₃;

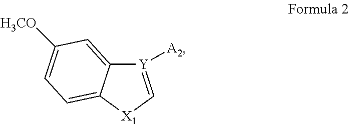

wherein A₂ is CH₂CH₂NHCOCH₃, and X₁ is S, O or CH₂ when Y is CH, or X₁ is NH when Y is N;

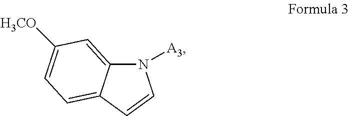

wherein A₃ is CH₂CH₂NHCOCH₃;

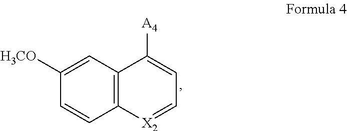

wherein A₄ is CH₂CH₂NHCOCH₃, and X₂ is CH or N;

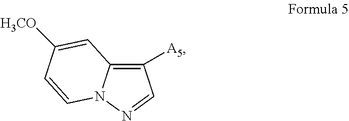

wherein A₅ is CH₂CH₂NHCOCH₃;

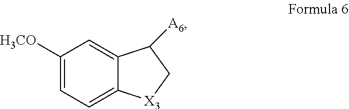

wherein A₆ is CH₂CH₂NHCOCH₃, and X₃ is NH or CH₂;

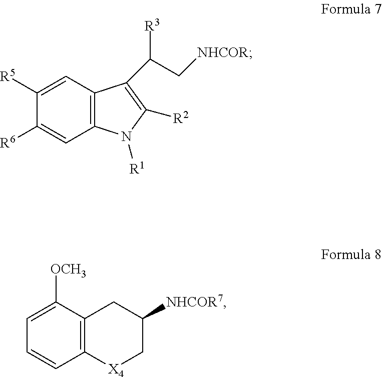

wherein X₄ is CH₂ when R⁷ is methyl, or X₄ is O when R⁷ is n-propyl;

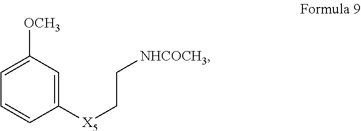

wherein X₅ is CH₂ or NCH₃;

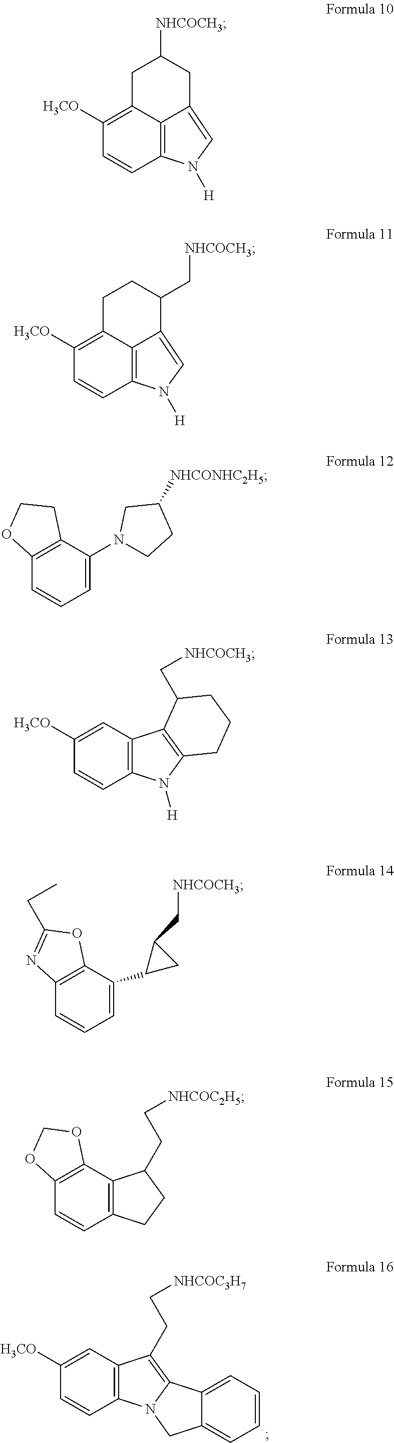

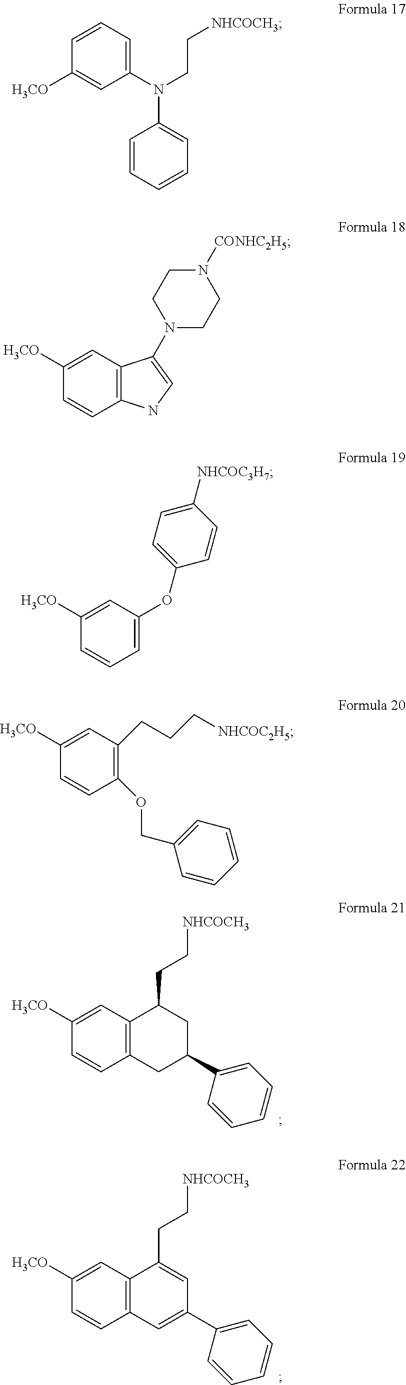

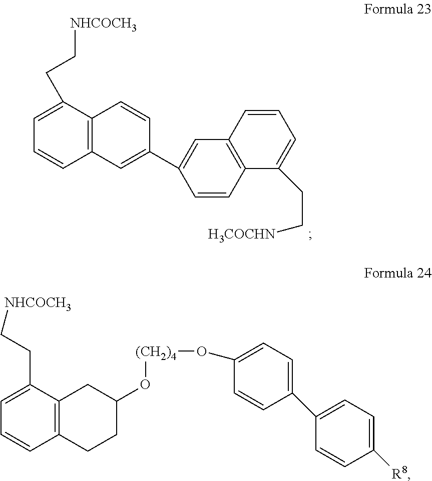

wherein R⁸ is COOH or CH₂OH;

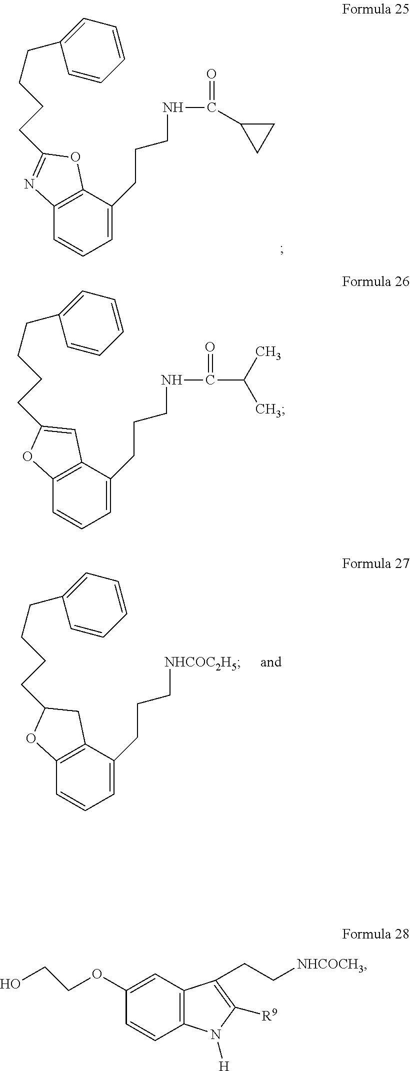

wherein R⁹ is H or Br.

According to the present inventive concept, the dosage of the composition comprising the melatonin associated compound may be appropriately determined according to various factors including the species, gender, body weight or age of the subject, the stage, symptom or severity of the disease, and the routes, timing or frequency of the administration.

The melatonin associated compound may be administered to the subject in an amount ranging from 0.0001 mg/day to 50 mg/day. In an embodiment, a lower limit of the dosage is 0.0005 mg/day, 0.001 mg/day, 0.05 mg/day, 0.1 mg/day, 0.15 mg/day, 0.2 mg/day, 0.5 mg/day, 1 mg/day or 2 mg/day, and an upper limit of the dosage is 50 mg/day, 25 mg/day, 15 mg/day, 12 mg/day, 10 mg/day, 8 mg/day or 5 mg/day. For example, the dosage of the composition may be from 0.001 mg/day to 50 mg/day, from 0.01 mg/day to 25 mg/day, from 0.05 mg/day to 10 mg/day, from 1 mg/day to 15 mg/day, from 3 mg/day to 20 mg/day, from 1 mg/day to 5 mg/day, and from 0.01 mg/day to 10 mg/day.

In an exemplary embodiment of the present disclosure, the melatonin associated compound may be administered to the subject in an amount of about 3 mg/day.

According to the present inventive concept, the administration of the composition may be conducted, for example, once per day, twice per day, 3 times per day, and 4 times per day. In an embodiment, the administration of the composition can be conducted twice per day.

According to the present inventive concept, the melatonin associated compound in the composition may be in an amount of from 0.001% to 30% by weight. In an embodiment, a lower limit of the amount is 0.005%, 0.01%, 0.05%, 0.1%, 0.2%, 0.5%, 0.8%, 1%, 3% or 5%, and an upper limit of the amount is 25%, 20%, 15%, 10%, 9%, 8%, 7% or 6%. For example, the amount of the melatonin associated compound in the composition may be from 0.1% to 10%, from 0.5% to 5%, from 1% to 8%, from 3% to 8% or from 3% to 5% by weight.

The composition comprising the melatonin associated compound also can be administered to the subject by any suitable route, including orally, intravenously, intraperitoneally, intramuscularly, externally, and transdermally. Further, the composition administered by such routes may be used in the above-specified amount as the standard. For example, in the case of external use (i.e., external treatment agent), the melatonin associated compound may be used in the form of a hydrophilic ointment, hydrophobic ointment, or a patch in an amount of from 0.1% to 10% by weight, based on the total weight of the formulation.

Besides, the composition comprising the melatonin associated compound may be administered by transdermal administration, nasal administration as well as those described in the U.S. Pat. No. 5,508,039 and Patent No. WO1998042333, which are incorporated herein by reference entirely.

In the exemplary embodiments of the present disclosure, the composition comprising the therapeutically effective amount of the melatonin associated compound is orally administered or topically administered.

In one embodiment of the present disclosure, the composition may be formulated in one form selected from the group consisting of a solution, a liniment, a lotion, a spray, an aerosol, an ointment, a foam, a cream, a gel, a paste, a patch, a glue, a film, a powder, a tablet, a pill, and a wound dressing.

In an embodiment, the composition of the present disclosure may further comprise a pharmaceutically acceptable excipient, wherein the pharmaceutically acceptable excipient may be a filler, a binder, a preservative, a disintegrating agent, a lubricant, a suspending agent, a wetting agent, a solvent, a surfactant, an acid, a flavoring agent, polyethylene glycol (PEG), alkylene glycol, sebacic acid, dimethyl sulfoxide, and an alcohol.

In an embodiment, the composition of the present disclosure may also include an adjuvant such as antioxidants, vitamins, and minerals. Exemplary vitamins that are useful as an adjuvant for the composition include, but are not limited to, Vitamin A (with carotenoids), thiamine (vitamin B1), riboflavin (vitamin B2), niacin (vitamin B3), pantothenic acid (vitamin B5), pyridoxine derivatives (vitamin B6), Cyanocobalamin (vitamin B12), Vitamin C, or Vitamin E. Exemplary minerals that are useful as an adjuvant include, but are not limited to, calcium, magnesium, sodium, selenium, copper, potassium and zinc.

According to the present inventive concept, the composition comprising the therapeutically effective amount of the melatonin associated compound may be administered to a subject in a period sufficient to ameliorate atopic dermatitis.

The period sufficient to ameliorate atopic dermatitis may depend on the species, gender, body weight or age of the subject, the stage, symptom or severity of the disease, and the routes, timing or frequency of the administration. The period may be, for example, lasting for 3, 4, 5 or 6 days, or 1, 2, 3 or 4 weeks or 1, 2, 3, 4, 6 months or even longer, as long as no side effect occurs during the treatment period. In the exemplary embodiments of the present disclosure, the period can be in a range of from 3 days to 2 years. In another embodiment, the period ranges from 2 weeks to 12 months. In yet another embodiment, the period ranges from 2 to 4 weeks.

According to the present inventive concept, the melatonin associated compound serves as the only active ingredient for atopic dermatitis in the composition.

In one embodiment, the composition may be administered to a subject in combination with another active ingredient unless the effect of the disclosure is inhibited. The melatonin associated compound and another active ingredient may be provided in a single composition or in separate compositions.

In an embodiment, the administration of the melatonin associated compound in the method provided by the present inventive concept may be combined with any suitable conventional therapy.

One of the suitable conventional therapies may be administration of therapeutic agents for AD. The therapeutic agents for AD can be, but not limited to, steroids, anti-histamine agents, such as cyproheptadine (Pilian®), cetirizine (Alltec®), loratadine (Lomidine®), and chlorpheniramine (Orolisin®); natural or synthetic immune inhibitors, such as tacrolimus and pimecrolimus.

Other suitable conventional therapies may include, but not limited to phototherapy, skin care, protection from allergen exposure, or administration of a skin moisturizer, an adjuvant, an antioxidant, a vitamin or a mineral, and the like.

The method provided by the present inventive concept further comprises an additional therapeutic agent for treating atopic dermatitis. Also, the method provided by the present inventive concept may be in combination with an additional therapy for preventing or treating atopic dermatitis.

When the method provided by the present inventive concept comprises the use of a melatonin associated compound and additional therapies (e.g., therapeutic agents for AD), the melatonin associated compound and the therapies may be administered simultaneously, separately or sequentially. The administration dose and frequency of the therapies may be determined based on the treatment results of the subject.

According to the present inventive concept, the method for treating AD can be used in a mammal, preferably a human.

When the method provided by the present inventive concept is used in human, the age of the subject may be in a range of from 0 to 65 years old, such as from 0 to 18, from 0 to 1, from 1 to 18, or from 1 to 65 years old.

Hereinafter, one or more exemplary embodiments of the present general inventive concept will be described in detail with reference to accompanying drawings. The examples below should not be taken as a limit to the scope of the present disclosure.

### EXAMPLE

### Example 1: Randomized Clinical Trial

### 1. Participants

A randomized clinical study with a double-blind, placebo-controlled crossover design was performed. The institutional review committee of National Taiwan University Hospital approved the study protocol. This study conformed to the principles of the Declaration of Helsinki. Patients or their guardians provided written informed consent.

Patients aged 1 to 18 years with physician-diagnosed AD involving at least 5% of the total body surface area were recruited from the pediatric and dermatology outpatient departments of the National Taiwan University Hospital from Aug. 1, 2012, through Jan. 31, 2013. Follow-up was completed on Apr. 13, 2013. Those patients with sleep problems occurring more than 3 days per week during the previous 3 months were eligible.

The term “sleep problem” used herein was referred as any difficulty with sleep initiation or maintenance that led to impaired quality of life or interfered with daytime activities for the child or for family members.

Seventy-three children with AD were recruited for this study. Baseline demographic and clinical characteristics are shown in Table 1. Besides, the patient's original treatments (such as additional medicaments or therapies) for AD, if any, can be maintained to be the same throughout the study period.

TABLE 1
Baseline Characteristics of the Patients at Randomization

| | | Patients by Treatment Group^{a} | |
|---|---|---|---|
| | All Patients | Melatonin | Placebo |
| Characteristic | (n = 48) | (n = 24) | (n = 24) |
| Female, No. (%) | 23 (48) | 13 (54) | 10 (42) |
| Age, y | 7.5 (3.7) | 7.6 (4) | 7.3 (3.5) |
| SCORAD index^{b} | 47.3 (22.7) | 48.0 (24.9) | 46.6 (20.7) |
| Objective SCORAD index^{c} | 37.0 (21.3) | 38.7 (22.8) | 35.2 (20.1) |
| Pruritus score^{d} | 5.6 (2.3) | 5.0 (2.4) | 6.3 (2.0) |
| Subjective sleep loss score^{d} | 4.7 (2.8) | 4.3 (2.4) | 5.1 (3.1) |
| Sleep-onset latency, min | 36.5 (39.9) | 41.8 (42.7) | 31.4 (37.2) |
| Sleep efficeiency, % | 77.4 (12.0) | 75.3 (12.3) | 79.3 (11.7) |
| WASO, min | 74.2 (44.5) | 73.5 (41.8) | 74.9 (47.8) |
| Wake in sleep interval, % | 15.2 (9.2) | 16.1 (9.7) | 14.4 (8.8) |
| Total sleep time, min/night | 415.8 (81.0) | 393.0 (88.1) | 437.7 (68.3) |
| Mobility sleep, % | 10.6 (6.9) | 11.6 (7.9) | 9.6 (5.9) |
| Fragmentation index^{e} | 15.6 (8.7) | 17.3 (9.2) | 14.0 (8.1) |
| Serum IgE level, kU/L | 2130.5 (3222.9) | 1882.6 (2451.7) | 2389.7 (3915.2) |
| Urinary 6-sulfatoxymelatonin level, ng/mL | 53.5 (62.7) | 54.0 (66.0) | 52.9 (61.1) |

SCORAD: Scoring Atopic Dermatitis;
WASO: wake after sleep onset.
^{a}Determined by which treatment was received first. Unless otherwise indicated, data are expressed as mean (SD).
^{b}Scores range from 0 to 103, with greater scores indicating worse symptoms.
^{c}Scores range from 0 to 83, with greater scores indicating worse symptoms.
^{d}Scores range from 0 to 10, with greater scores indicating worse symptoms.
^{e}Greater scores indicate more fragmented sleep.

### 2. Randomization and Masking

The study participants were randomly assigned to 2 groups in a 1:1 ratio, with a block size of 4. One group received melatonin first and then placebo, and the other group received placebo first and then melatonin. The melatonin and placebo tablets were identical in appearance. The participants and their caregivers, treating physicians, those assessing outcomes, and those analyzing the data were all masked to group assignment.

### 3. Study Design

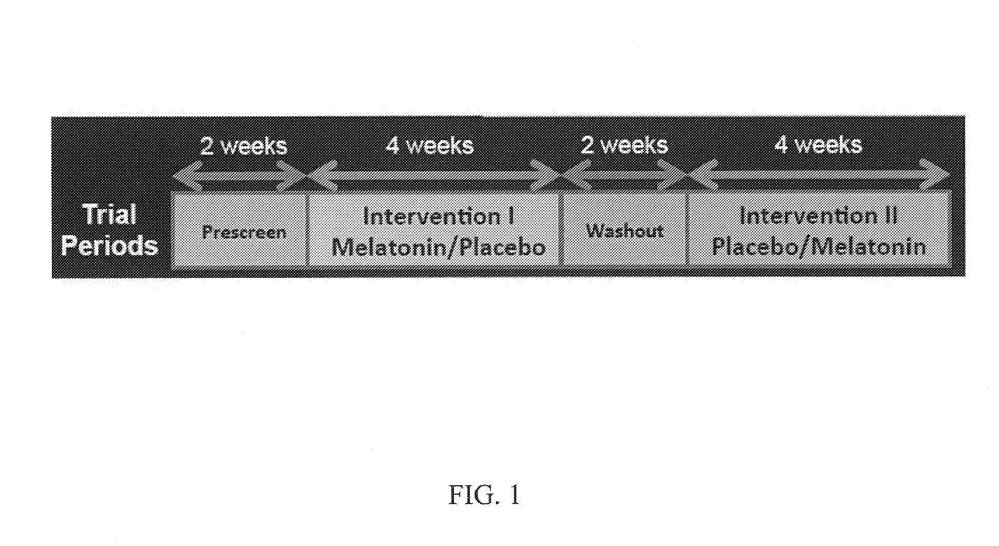

As shown in FIG. 1, the participants first underwent a 2-week prescreening period in which they kept a sleep diary, adhered to a fixed sleep schedule, and avoided caffeinated drinks. They were then randomized to receive oral melatonin, 3 mg/d (General Nutrition Corporation), or placebo (Standard Chem & Pharm Co, Ltd) at bedtime for 4 weeks. After a 2-week washout period, participants crossed to the alternate treatment.

Visits occurred at the start of the prescreening and on the first and last day of each treatment period, during which AD disease severity was assessed, blood and urine samples were collected, questionnaires evaluating subjective symptoms were filled out, and pill count and adverse events were recorded. No patient withdrew owing to adverse events, and no adverse event was reported throughout the study.

### 4. Assessment Index

In this clinical trial, the primary study outcome was the Scoring Atopic Dermatitis (SCORAD) index for the evaluation of AD severity. Secondary outcomes included the objective sleep variables measured by actigraphy, the patient's subjective description of the change in sleep and dermatitis severity, the sleep variables measured by polysomnography, nocturnal urinary levels of 6-sulfatoxymelatonin, and serumtotal and allergen-specific IgE levels. These assessment indexes recoded in this study were shown in Table 1.

### <Severity of AD>

Severity of AD was assessed using the SCORAD index (range, 1-103, with greater scores indicating worse symptoms) by the same physician (Y.-S.C.) blinded to the treatment randomization. The SCORAD index includes subjective visual analog scale scores from 0 to 10 (with greater numbers indicating worse symptoms) for degree of pruritus and sleep loss, which are referred to as the pruritus score and the subjective sleep score, respectively, in this study. The objective SCORAD index, which excludes the SCORAD score for subjective symptoms (range, 0-83, with greater scores indicating worse symptoms), was also used for analysis.

### <Sleep Evaluation>

Sleep was evaluated by actigraphy (Mini-Mitter Actiwatch; Philips-Respironics). Each participant wore the actigraphic device on the nondominant wrist for 3 consecutive nights starting 3 nights before each treatment period and for the last 3 nights of each treatment period. In a subgroup of patients who offered consent, polysomnography was performed on the night before the first day and on the last night of each treatment period.

### <Biochemical Index>

The first urine sample on the morning after each actigraphic examination was obtained. Levels of urinary 6-sulfatoxymelatonin were assayed by enzyme-linked immunosorbent assay with a commercialized kit (IBL International GmbH) and were used to represent the melatonin level throughout the previous night.

A peripheral blood sample was taken from each participant at 9 AM on the morning after the last night of the sleep examination and stored at −80° C. until measurements were performed.

The serum total IgE level and levels of allergen-specific IgE to Dermatophagoides pteronyssinus (Derp), Dermatophagoides farinae (Derf), Staphylococcus aureus enterotoxin A, and S aureus enterotoxin B were measured with a fluorescence enzyme immunoassay (Immuno-CAP; Phadia AB). Allergen-specific IgE levels higher than 0.35 kU/L were defined as positive.

### Example 2: AD Severity Amelioration by Administering Melatonin

Regarding AD severity, the melatonin group had a greater decrease in the mean (SD) SCORAD index than the placebo group in the first treatment period (from 49.5 to 42.3 vs from 49.0 to 48.8). Similar result is found in the second treatment period (from 48.7 to 38.3 vs from 46.8 to 44.6).

As shown in Table 2, melatonin significantly improved AD severity by a mean reduction of the SCORAD index by 9.9 in 4 weeks. In addition, linear mixed-effects models were used to compare the mean change in outcomes after vs before treatment between the melatonin and placebo phases, and the linear mixed-effects model showed that after adjusting for age and sex, melatonin treatment resulted in a decrease of the SCORAD index by 9.1 compared with placebo.

TABLE 2
Effects of Melatonin vs Placebo

| | | | | | | | Difference of Treatment | | |
|---|---|---|---|---|---|---|---|---|---|
| | Results by Study Phase, Mean (SD) | | | | | | Effect From the Linear | | |
| | | | | | | | Mixed-Effects Model^{a} | | |
| | Melatonin | | | Placebo | | | Mean | P | P |
| Measure | Before | After | Difference | Before | After | Difference | (95% CI) | Value | Value^{b} |
| SCORAD | 49.1 | 40.2 | −9.9 | 48.0 | 46.8 | −0.7 | −9.1 | <.001 | <.001 |
| index^{c} | (24.3) | (20.9) | (7.6) | (21.9) | (20.2) | (8.6) | (−13.7 to −4.6) | | |
| Objective | 38.6 | 31.3 | −8.2 | 37.4 | 37.6 | −0.6 | −8.7 | <.001 | <.001 |
| SCORAD | (22.1) | (19.2) | (7.2) | (20.8) | (19.4) | (6.3) | (−12.6 to −4.8) | | |
| index^{d} | | | | | | | | | |
| Pruritus | 5.6 | 5.1 | −0.6 | 5.7 | 5.1 | −0.5 | 0.0 | .99 | .70 |
| score^{e} | (2.5) | (2.5) | (2.0) | (2.1) | (2.4) | (2.9) | (−1.2 to 1.2) | | |
| Subjective | 4.8 | 3.8 | −1.1 | 4.9 | 4.1 | −0.7 | −0.4 | .57 | .54 |
| sleep score^{c} | (2.7) | (2.4) | (2.5) | (2.6) | (2.5) | (2.8) | (−1.6 to 0.9) | | |
| Sleep-onset | 44.9 | 21.6 | −23.4 | 31.5 | 29.6 | −1.2 | −21.4 | .02 | .005 |
| latency, min | (39.5) | (20.4) | (36.1) | (28.3) | (23.8) | (31.5) | (−38.6 to −4.2) | | |
| Sleep | 74.4 | 76.7 | 2.4 | 78.0 | 77.7 | 0.6 | 2.2 | .41 | .74 |
| efficiency, % | (12.5) | (12.8) | (13.0) | (11.0) | (10.1) | (9.2) | (−3.1 to 7.4) | | |
| WASO, min | 83.6 | 74.5 | −9.0 | 74.0 | 75.9 | −2.3 | −8.2 | .49 | .91 |
| | (63.8) | (53.5) | (68.5) | (53.5) | (46.9) | (38.1) | (−31.9 to 15.4) | | |
| Wake in sleep | 17.6 | 15.6 | −2.1 | 14.8 | 15.7 | −0.2 | −2.3 | .33 | .59 |
| interval, % | (11.6) | (10.7) | (13.1) | (10.3) | (9.7) | (7.4) | (−6.8 to 2.3) | | |
| Total sleep time, | 380.1 | 390.1 | 10.0 | 410.5 | 391.7 | −10.5 | 24.8 | .21 | .20 |
| min/night | (87.0) | (73.8) | (80.3) | (73.0) | (89.2) | (88.2) | (−14.5 to 64.2) | | |
| Mobility | 11.6 | 9.7 | −2.0 | 9.5 | 10.4 | 0.9 | −2.9 | .05 | .21 |
| in sleep, % | (7.3) | (4.7) | (7.6) | (4.0) | (4.6) | (4.4) | (−5.9 to 0.0) | | |
| Fragmentation | 16.0 | 13.6 | −2.4 | 14.7 | 15.3 | 0.7 | −3.2 | .09 | .40 |
| index^{f} | (7.8) | (5.8) | (9.2) | (5.5) | (6.6) | (6.9) | (−6.8 to 0.5) | | |
| IgE level, | 2337.2 | 2483.8 | 108.4 | 2457.1 | 2755.9 | 243.6 | −113.6 | .57 | .56 |
| kU/L | (3327.5) | (3685.2) | (463.2) | (3585.3) | (4184.6) | (900.7) | (−511.2 to 283.9) | | |

SCORAD: Scoring Atopic Dermatitis; WASO: wake after sleep onset.
^{a}Estimated difference of the effect of melatonin vs placebo by the linear mixed model, adjusted for age and sex (coefficient of the phase × time interaction term).
^{b}Comparisons of the absolute difference from baseline after melatonin and placebo treatment by the Wilcoxon signed rank test.
^{c}Scores range from 0 to 103, with greater scores indicating worse symptoms.
^{d}Scores range from 0 to 83, with greater scores indicating worse symptoms.
^{e}Scores range from 0 to 10, with greater scores indicating worse symptoms.
^{f}Greater scores indicate more fragmented sleep.

More patients subjectively believed that their dermatitis had improved after melatonin treatment compared with placebo (17 of 36 [47%] vs 12 of 38 [32%]).

To avoid confounding by the patient's subjective scores of pruritus and sleeplessness, the objective SCORAD index, which represents the degree of severity based on the skin, was also analyzed.

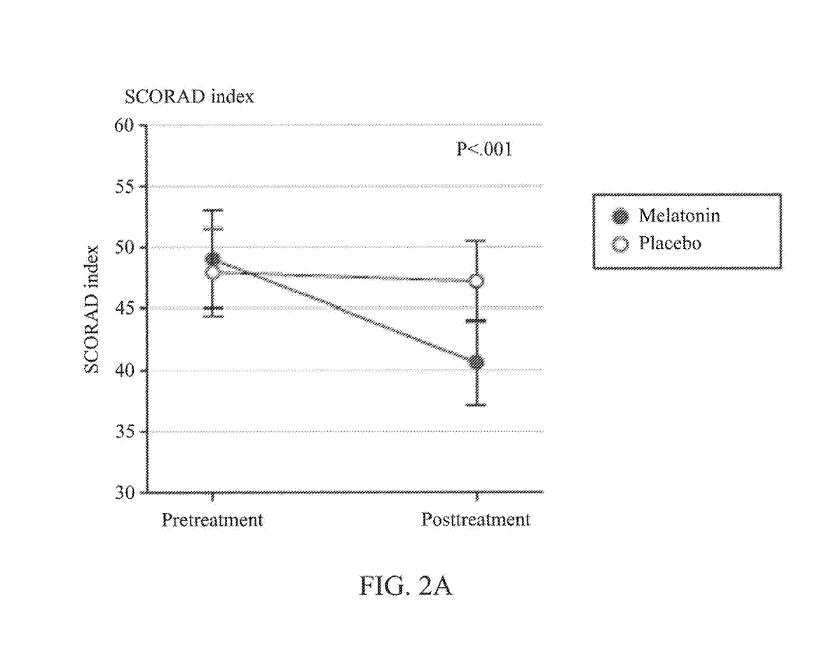

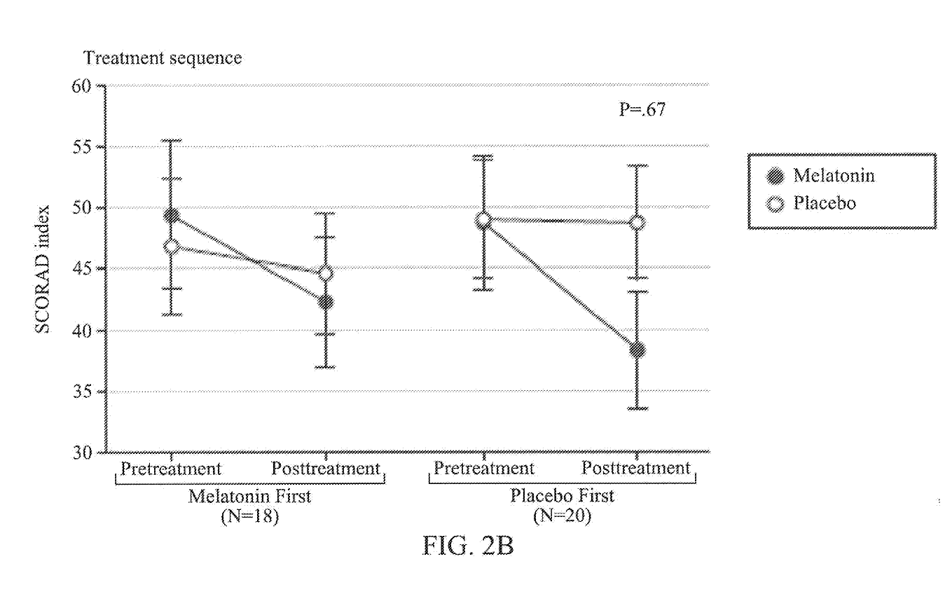

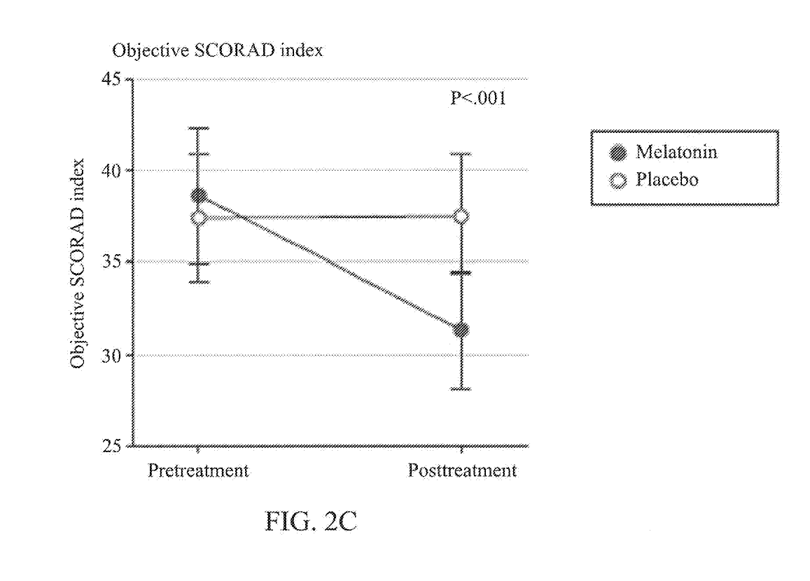

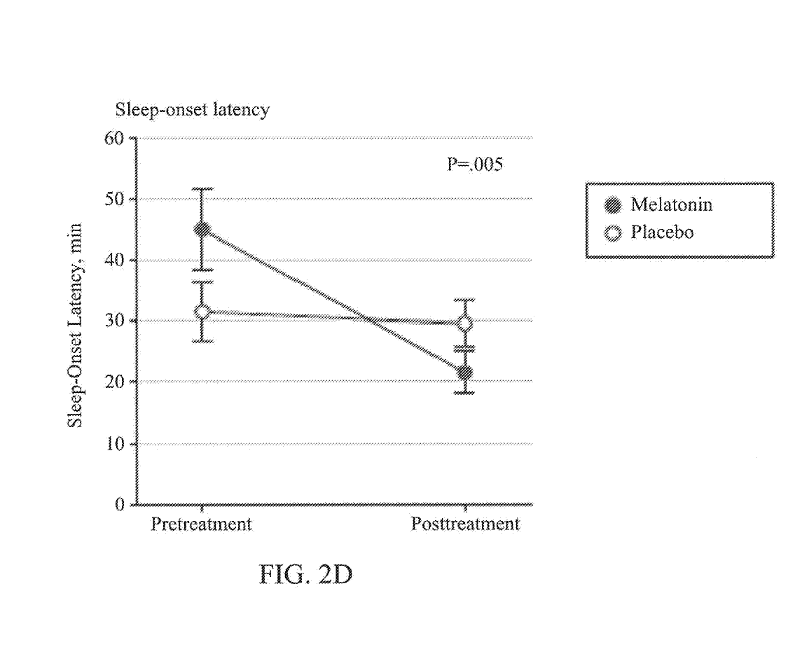

As shown in Table 2 and FIG. 2B, melatonin treatment also resulted in a decrease of the objective SCORAD index by 8.7 compared with placebo, with no significant carryover effect (P=0.81) and a significant period effect (P=0.02). The term “carryover effect” used herein was defined as a statistically significant sequence effect.

Wilcoxon signed rank test as a sensitivity analysis was also performed to compare the difference of each outcome measure before and after treatment between melatonin and placebo. As shown in FIGS. 2A and 2C, the Wilcoxon signed rank test yielded results consistent with the linear mixed-effects model. Melatonin treatment apparently decreased the SCORAD index than placebo.

### Example 3: Effect of Administration of Melatonin on Sleep Disturbance for Subject with AD

Evaluation of sleep variables measured by actigraphy showed that the melatonin group had a greater decrease in the sleep-onset latency than the placebo group in the first treatment period (from 42.8 to 24.4 minutes vs from 34.5 to 22.9 minutes). Similar result is found in the second treatment period (from 46.7 to 19.1 minutes vs from 27.9 to 37.5 minutes). Also, more patients believed that their sleep had improved after melatonin treatment compared with placebo (17 of 37 [46%] vs 13 of 38 [34%]).

As shown in Table 2, the linear mixed-effects model showed that after adjusting for age and sex, melatonin treatment resulted in a decrease of the sleep-onset latency by 21.4 minutes more than placebo. Besides, no significant carryover effect or period effect was found in the experiment. The effect on the other sleep variables measured by actigraphy did not differ significantly between the melatonin and placebo phases.

As shown in FIG. 2D, the results from the Wilcoxon signed rank test arrived at the same conclusions. Melatonin treatment decreased the sleep-onset latency significantly, while the sleep-onset latency almost unchanged in placebo treatment during the treatment period.

The reduction in the sleep-onset latency did not correlate significantly with the degree of improvement in the SCORAD index with melatonin treatment (r=−0.04; P=0.85).

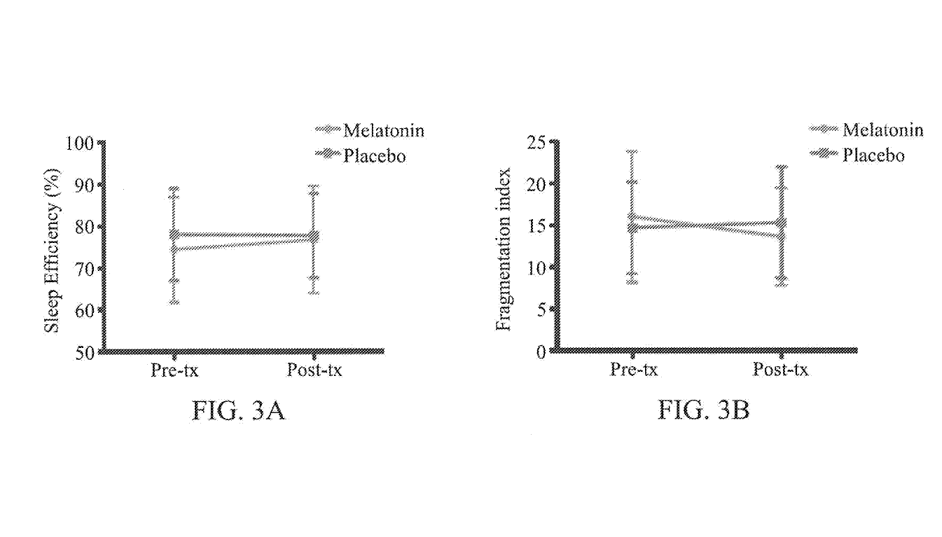

Melatonin and placebo treatment did not have a significantly different effect on the stages of sleep and limb movement (P>0.05) for those patients underwent polysomnography in addition to actigraphy. As shown in FIG. 3, compared melatonin treatment to placebo, no significant effect was found on sleep efficiency, wake time during sleep, or sleep fragmentation.

### Example 4: Effect of Administration of Melatonin on Biochemical Index

Nocturnal urinary 6-sulfatoxymelatonin levels significantly increased after treatment with melatonin compared with placebo (P<0.001) (See Table 1), although there was no significant difference between the effects of melatonin and placebo on total IgE level or on allergen-specific IgE levels to Derp, Derf, or S aureus enterotoxins A or B (P>0.05) (See Table 2).

The most commonly reported adverse effects of melatonin included drowsiness, dizziness, nausea, and headaches, but the occurrence of these events did not differ significantly between melatonin and placebo. In fact, no adverse effect of medication was reported throughout the study.

The exemplary embodiments of the present disclosure demonstrate that the oral melatonin associated compound, such as melatonin, significantly improved the disease severity in children and adolescents with AD. Besides, the melatonin associated compound treatment significantly shortens sleep-onset latency by a mean of 23.4 minutes (52.1%). It means that the melatonin associated compound has dual effect on the improvements of AD and sleep disturbance.

Besides, the exemplary embodiments of the present disclosure show evidence of improvement of sleep by melatonin administration in children with AD by using a validated objective measuring tool and provide evidence of good safety profile of melatonin.

The exemplary embodiments of the present disclosure also demonstrate that no significant correlation between improvement in the SCORAD index and reduction in the sleep-onset latency. Furthermore, melatonin did not improve the sleep variables other than sleep-onset latency, including sleep efficiency, mobile percentage in sleep, sleep fragmentation, and sleep architecture. Therefore, the effect of melatonin on AD might not be attributable to its effect on sleep but instead through its immunomodulatory or antioxidative properties.

Both the first treatment period and the second treatment period show the effect of melatonin on improvement of dermatitis severity. This result supports a true effect of melatonin on improvement of dermatitis severity in children with AD.

From the above, the melatonin associated compound significantly improved the severity of dermatitis and reduced sleep-onset latency in children with AD and sleep disturbance. The melatonin associated compound administration/supplementation is recommended for these patients because it is a potentially safe and effective way to improve their sleep and skin condition simultaneously.

### Example 5: Effect of Topical Administration of Melatonin in a DNCB-Induced AD Mouse Model

### 1. Animals

Four- to six-week-old Balb/c mice were maintained under specific pathogen-free conditions. Animals were housed in an air-conditioned animal room (25±1° C.; relative humidity 40±5%) and were fed a laboratory diet and distilled water. Animal care and handling protocols were approved by the Animal Committee of the College of Medicine, Taiwan University, and all procedures were conducted in accordance with the U.S. National Institute of Health guidelines.

### 2. Topical Melatonin Treatment in 2,4-Dinitrochlorobenzene (DNCB)-Induced AD Mouse Model

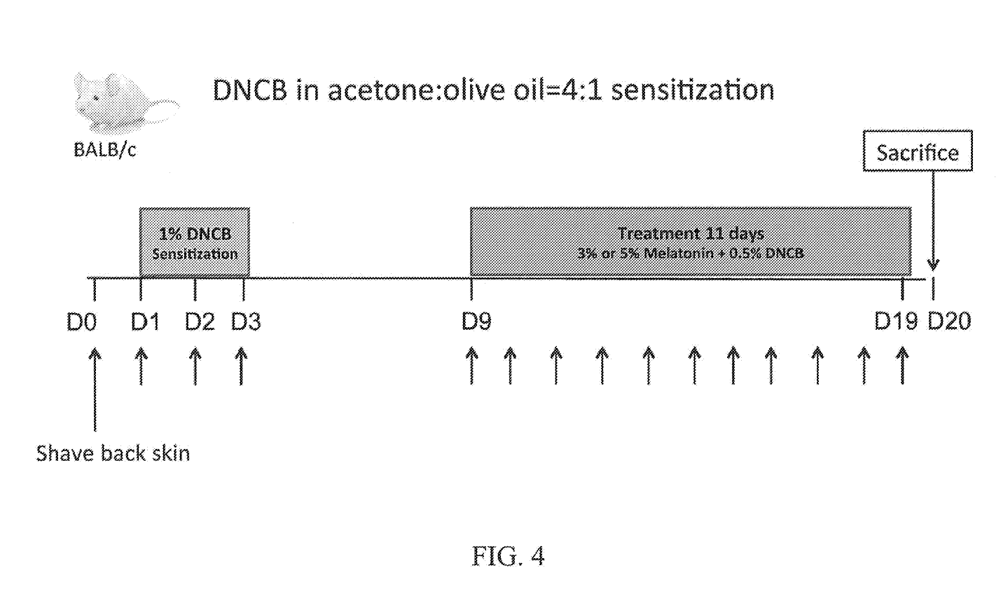

The protocol for establishing an AD mouse model in this Example was modified from those described previously. [10] Briefly, referring to FIG. 4, DNCB-induced AD mouse model was prepared by the repeated applications of 20 μL of 1% DNCB in acetone/olive oil (4:1) to the outer and inner surfaces of mouse ears and 100 μL of the same solution to the shaved back skin once on days 1, 2, and 3. Mice of Control group were treated with the identical volumes of vehicle.

The DNCB-sensitized mice were randomly assigned to one of three groups: DNCB (Positive control), DNCB+3% topical melatonin (Treatment: low-dose), DNCB+5% topical melatonin (Treatment: high-dose). Each group (including Control group) consisted of 5 mice. From days 9 to 19, sensitized mice were challenged by applying 0.5% DNCB to the skin surfaces. For the two groups treated with topical melatonin groups, 0.3 mg of topical melatonin ointment (3% or 5% melatonin in Vaseline) was given together with DNCB during the challenge period. For DNCB group, 0.3 mg of Vaseline was given together with DNCB during the challenge period. For Control group, 0.3 mg of Vaseline was given from days 9 to 19. This experiment was ended at Day 20.

### 3. Dermatitis Severity Evaluation

Macroscopic assessment of the severity of dermatitis was determined by a scoring method as described previously. [11] Briefly, the degree of each sign were scored as 0 (none), 1 (mild), 2 (moderate), and 3 (severe). This scoring was based on the severity of erythema (hemorrhage), edema, excoriation (erosion), and dryness (scaling). The total score (minimum 0 and maximum 12) from the four symptoms of each mouse were taken as the score for that mouse. Ear thickness was measured with a thickness gauge (Digimatic Indicator, Mitsutoyo, Tokyo). This assessment was performed on the last day of the experiment (Day 20) by an investigator who was blind to the grouping of the animals.

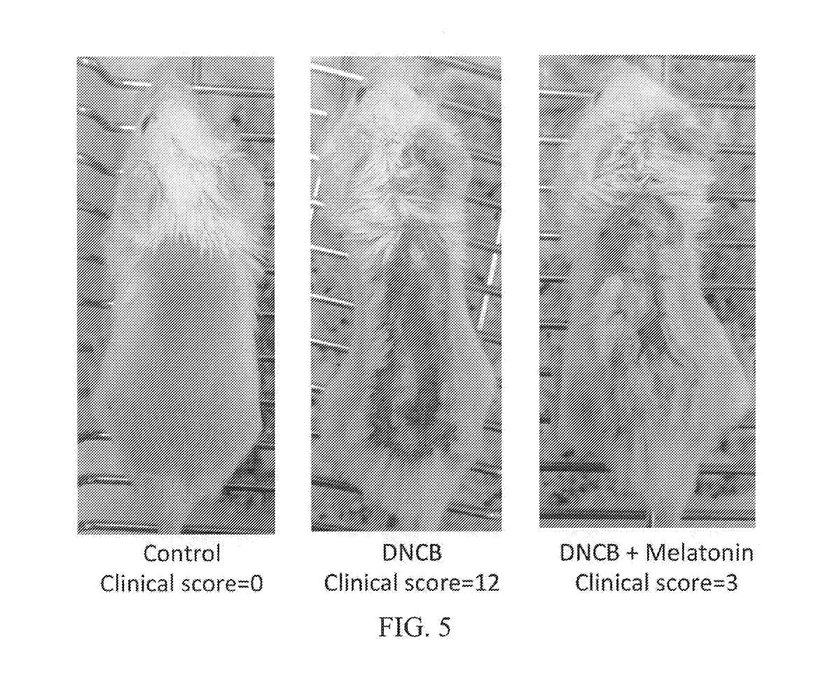

The results were shown in FIG. 5. In the DNCB-stimulated group (representing AD), severe skin erythema, skin excoriations with exudate, and poor hair growth were noted compared with Control group. In DNCB-stimulated mice treated with melatonin, dermatitis was improved, with less skin erythema, less skin excoriation, and improved hair growth. Dermatitis clinical score decreased from 12 to 3 after melatonin treatment.

### 4. Histological Examination

At the end of the experiment (Day 20), all the experimental animals were subjected to euthanization by carbon dioxide and the skin samples were collected for subsequent immunological and histological examinations. The skin of each mouse were fixed with 10% neutral-buffered formalin, and embedded in paraffin. Then, 4-μm-thick sections were cut and transferred onto slides. Deparaffinized skin sections were stained with hematoxylin and eosin (H&E) before they were examined at 200× magnification for evaluation of edema and polymorphonuclear leukocytes (PMN) number. To detect mast cell infiltration, deparaffinized skin sections were stained with toluidine blue. Polyclonal rabbit antibody against CD3 was used to stain T cells. The numbers of mast cells and T cells per 10 high power fields (HPF) were counted.

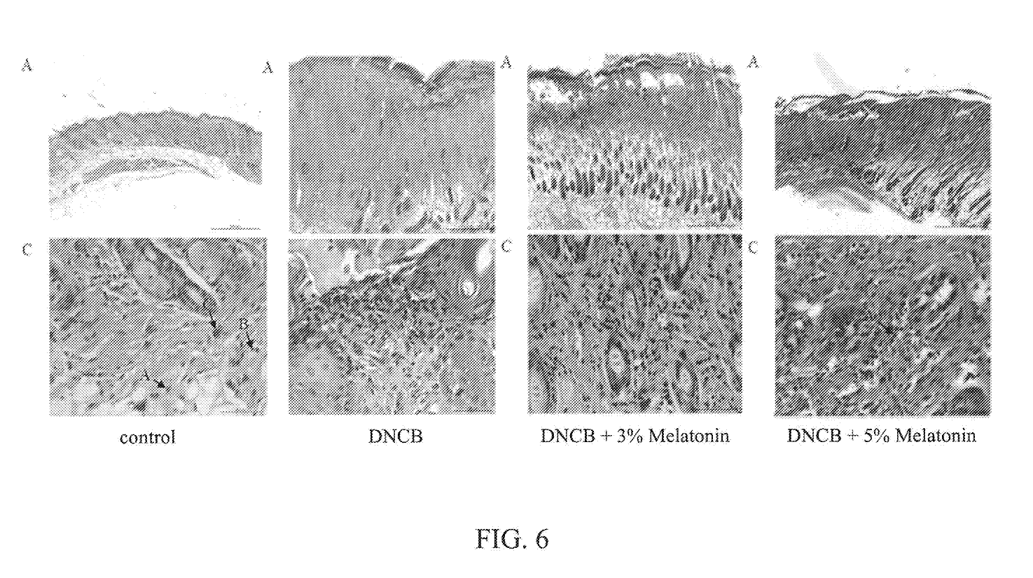

As shown in FIG. 6, the results based on H&E staining of the skin from DNCB-stimulated mice showed severe inflammation with infiltration of lymphocyts, neutrophils and mast cells, and severe epidermal hyperplasia. Both the 3% and 5% melatonin treatment group showed improved epidermal hyperplasia and less infiltration of lymphocytes.

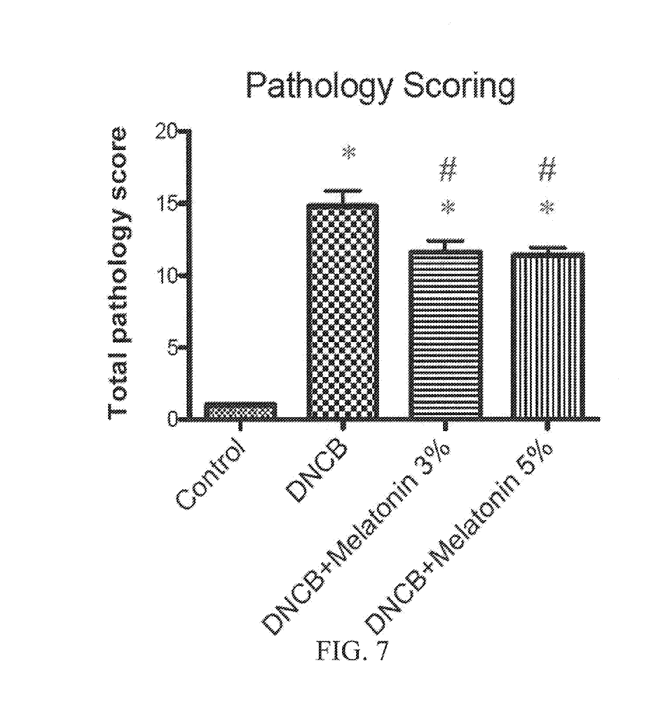

Further, degree of histopathological lesions of skin from each mouse was graded from one to five depending on severity: 1=minimal (<1%); 2=slight (1-25%); 3=moderate (26-50%); 4=moderate/severe (51-75%); and 5=severe/high (76-100%). The results were shown in FIG. 7 and Table 3. There was significant improvement in pathology scoring between 3% melatonin and DNCB group (p=0.04), and 5% melatonin and DNCB group (p=0.02).

TABLE 3
Summary of pathological incidence of skin in mice

| | | Group | | | |
|---|---|---|---|---|---|
| | | | Positive | Treatment | Treatment |
| Organ | Histopathological lesions | Control | control | (low-dose) | (high-dose) |
| Skin | Inflammation, neutrophil, lymphocyte and mast | 5/5 | 5/5 | 5/5 | 5/5 |
| | cell, epidermis, dermis and subcutis, minimal | 1.0 ± 0.0 | 4.2 ± 0.8 | 3.8 ± 0.4 | 3.8 ± 0.4 |
| | to severe/high | | | | |
| | Hyperplasia, epidermis and follicular epithelium, | 0 | 5/5 | 5/5 | 5/5 |
| | slight to moderate/severe | | 3.2 ± 0.8 | 2.4 ± 0.5 | 2.0 ± 0.0 |
| | Serocellular crust/parakeratosis, epidermis, slight | 0 | 5/5 | 5/5 | 5/5 |
| | | | 2.0 ± 0.0 | 2.0 ± 0.0 | 2.0 ± 0.0 |
| | Hyperplasia, sebaceous gland and hair follicle, | 0 | 5/5 | 5/5 | 5/5 |
| | dermis and subcutis, slight to moderate | | 2.2 ± 0.4 | 2.0 ± 0.0 | 2.4 ± 0.5 |
| | Infundibular hyperkeratosis, hair follicle, dermis, | 0 | 4/5 | 3/5 | 0 |
| | minimal to slight | | 1.2 ± 0.8 | 0.8 ± 0.8 | |
| | Necrosis, epidermis, slight | 0 | 1/5 | 0 | 1/5 |
| | | | 0.4 ± 0.9 | | 0.4 ± 0.9 |
| | Vacuolization, epidermis, minimal to slight | 0 | 5/5 | 3/5 | 4/5 |
| | | | 1.2 ± 0.4 | 0.6 ± 0.5 | 0.8 ± 0.4 |
| | Pustule, epidermis, slight | 0 | 1/5 | 0 | 0 |
| | | | 0.4 ± 0.9 | | |
| | Cumulative histological scores | 1.0 ± 0.0 | 14.8 ± 2.4 | 11.6 ± 1.8 | 11.4 ± 1.1 |

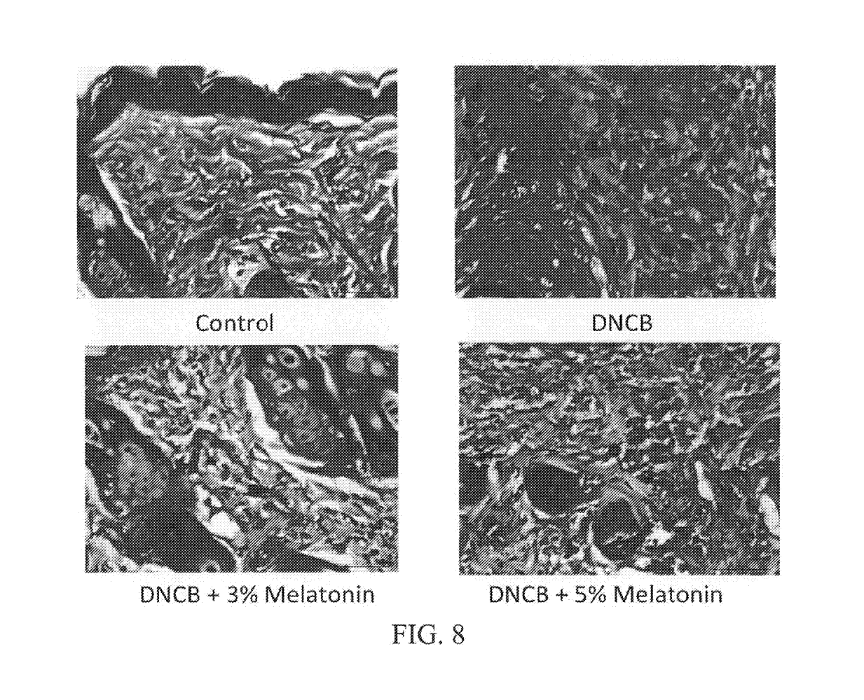

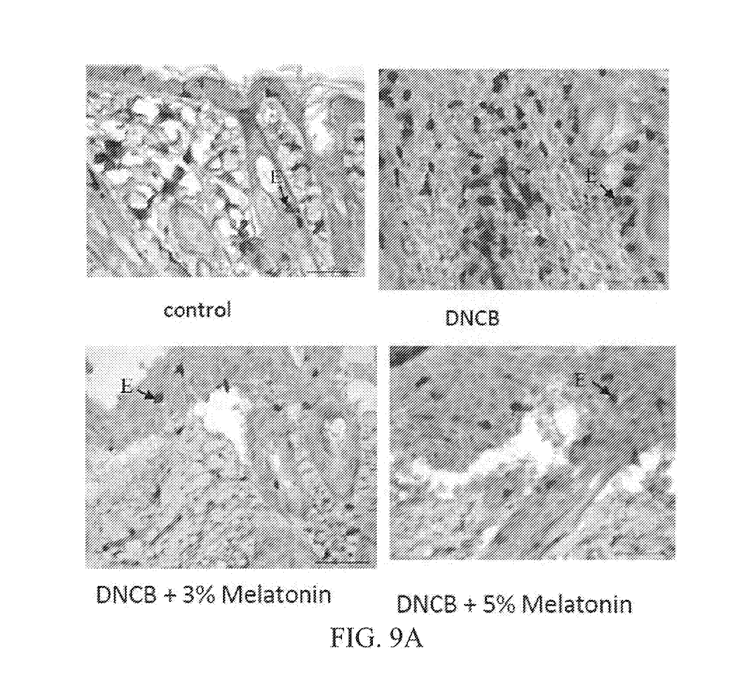

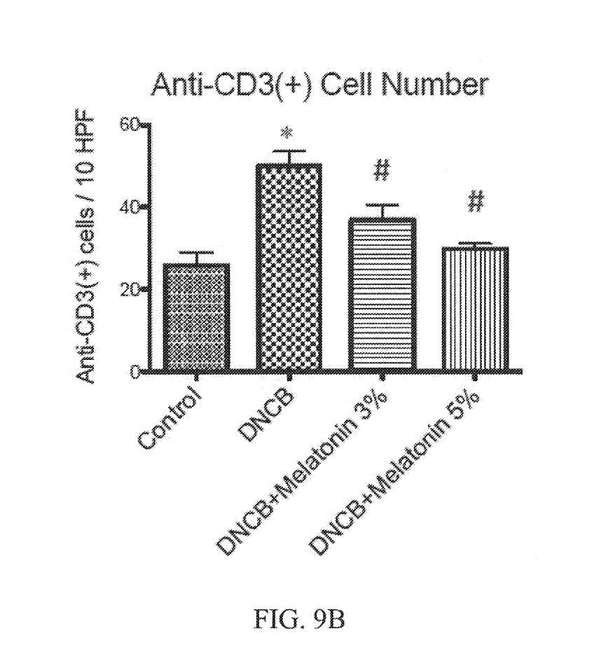

Referring to FIG. 8, it showed that mast cell infiltration increased after stimulation by DNCB compared with Control, and decreased after melatonin treatment. FIGS. 9A and B showed the results of immunohistochemistry staining for anti-CD3 positive cells (T lymphocytes), indicating that the number of anti-CD3 positive cells per 10 HPF under the microscope was significantly reduced after either 3% or 5% topical melatonin treatment compared with DNCB group (p=0.03 for 3% melatonin compared with DNCB group, and p=0.0007 for 5% melatonin compared with DNCB group).

### 5. IgE and Cytokine Measurement

Blood and dorsal skin of each mouse were collected on the last day of the experiment (Day 20) and stored at −80° C. until use. Serum IgE levels were measured using the ImmunoCAP fluorescence enzyme immunoassay (Phadia AB, Sweden) according to the manufacturer's instructions. The dorsal skin mRNA expression of the cytokines IL-4, IL-13, and IL-17 were measured by RT-PCR.

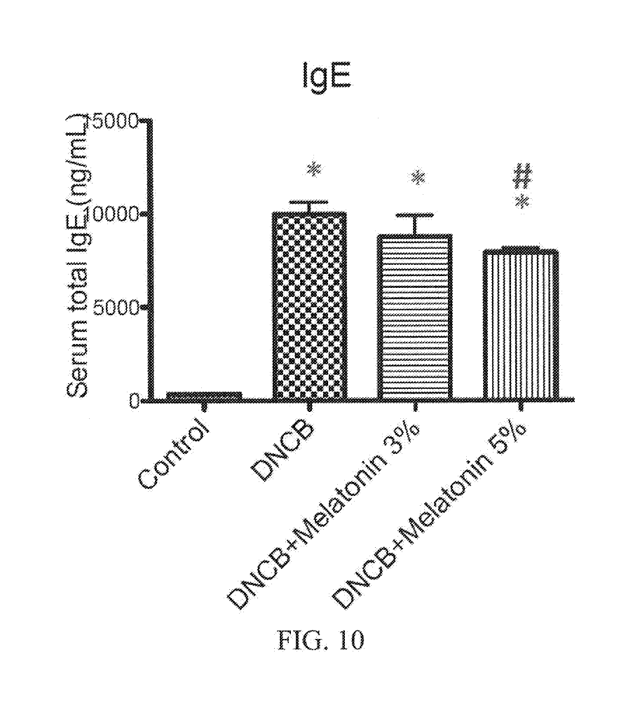

Referring to FIG. 10, the serum total IgE level significantly reduced after 5% melatonin treatment compared with the DNCB-stimulated group (p=0.02).

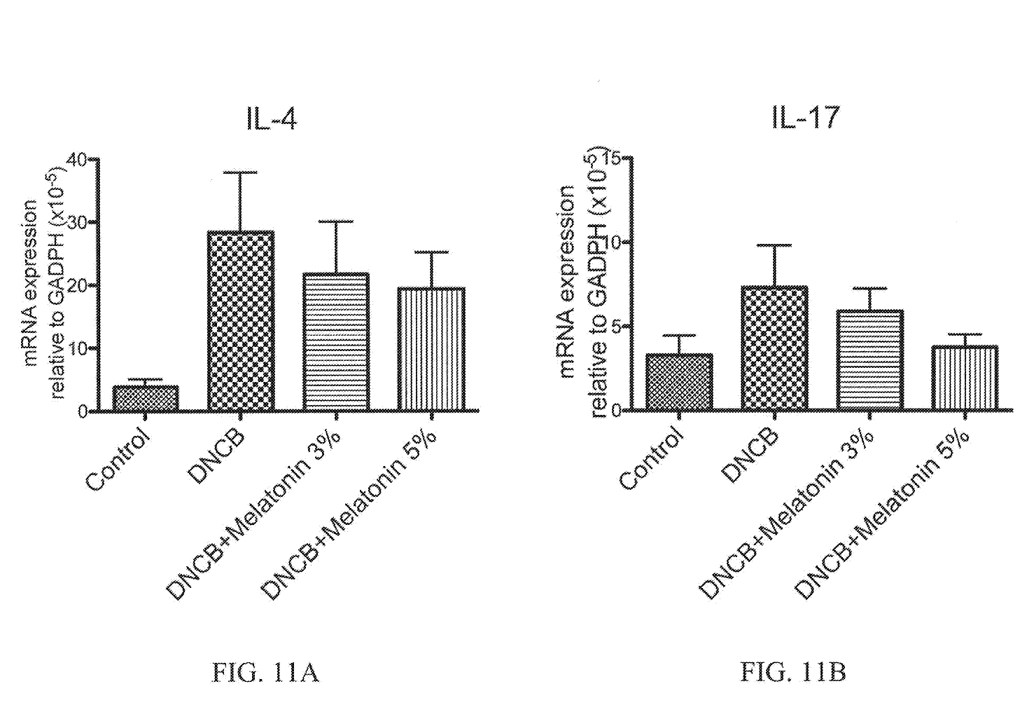

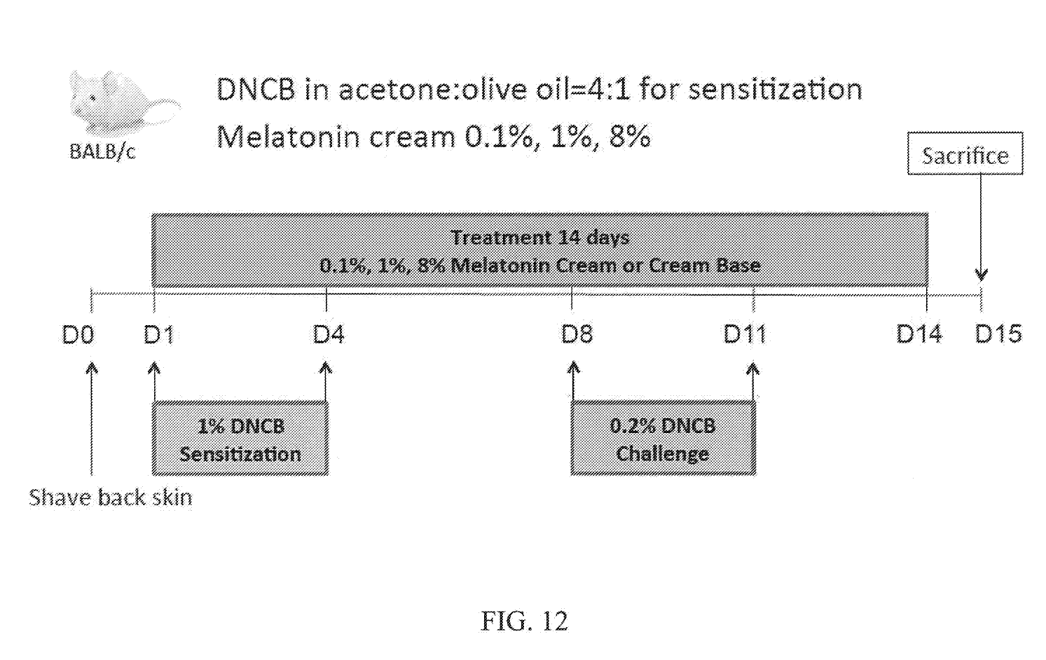

Referring to FIGS. 11A and 11B, the skin mRNA expression showed decreased for IL-4 and IL-17 after topical melatonin treatment. A dose-response effect was noted, as the IL-4 and IL-17 decreased more after 5% melatonin treatment compared with 3% melatonin treatment.

### 6. Statistical Analysis

Average data were presented as mean±SD. It was performed statistical analysis using IBM SPSS for MAC, version 20. Comparisons between different experimental groups were conducted using ANOVA followed by Bonferroni test. Statistical difference was considered significant when p<0.05.

### Example 6: Effect of Topical Administration of Different Dosage of Melatonin on Treatment of AD

### 1. Animals

Four- to six-week-old Balb/c mice were maintained under specific pathogen-free conditions. Animals were housed in an air-conditioned animal room (25±1° C.; relative humidity 40±5%) and were fed a laboratory diet and distilled water. Animal care and handling protocols were approved by the Animal Committee of the College of Medicine, Taiwan University, and all procedures were conducted in accordance with the U.S. National Institute of Health guidelines.

### 2. Topical Melatonin Treatment in DNCB-Induced AD Mouse Model

The protocol for establishing an AD mouse model in this Example was modified from those described previously. [12] Briefly, referring to FIG. 12, DNCB-induced AD mouse model was prepared by the repeated applications of 20 μL of 1% DNCB in acetone/olive oil (4:1) to the outer and inner surfaces of mouse ears and 100 μL of the same solution to the shaved back skin once on days 1 and 4. Mice of Control group were treated with the identical volumes of vehicle.

The DNCB-sensitized mice were randomly assigned to one of four groups: DNCB, DNCB+0.1% topical melatonin, DNCB+1% topical melatonin, DNCB+8% topical melatonin. Each group (including Control group) consisted of 3 mice. On days 8 and 11, sensitized mice was challenged by applying 0.2% DNCB to the skin surfaces. For the three groups treated with topical melatonin groups, 0.1 mg of topical melatonin cream was given twice a day from day 1 to day 14. The cream base consisted of a mixture of tefose 63, octyldodecyl myristate, sorbic acid, methyl paraben sodium, ddH₂O, and apricot kernel oil. For Control and DNCB groups, 0.1 mg of cream base was given twice a day from day 1 to day 14. This experiment was ended at Day 15.

### 3. Dermatitis Severity Evaluation

The dermatitis severity of each mouse treated with or without melatonin was evaluated in the same manner as those described in Example 5. This evaluation was performed on the last day of the experiment (Day 15) by an investigator who was blind to the grouping of the animals.

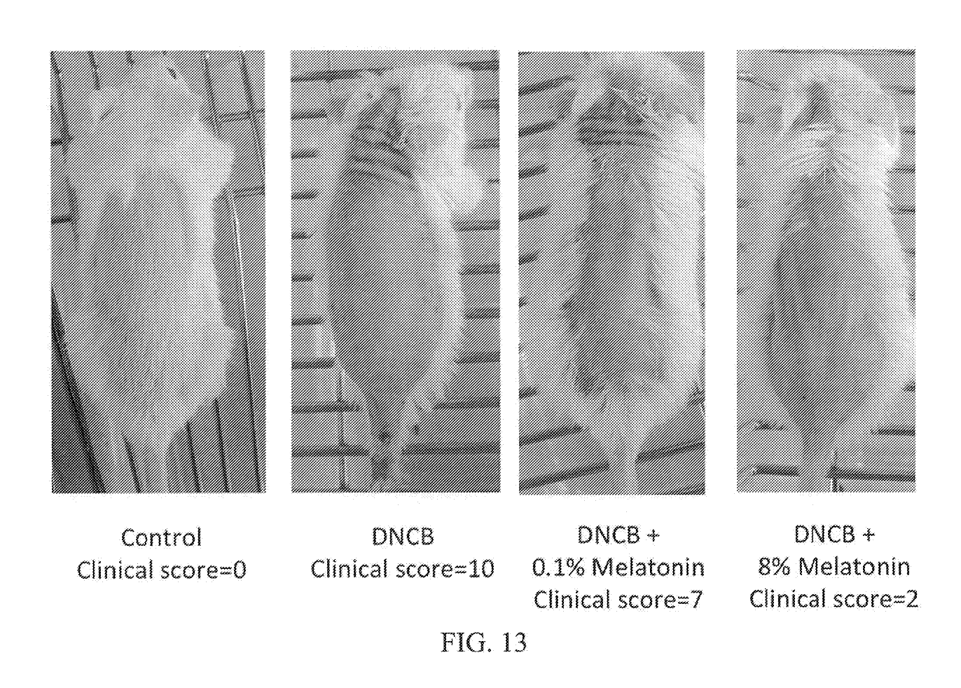

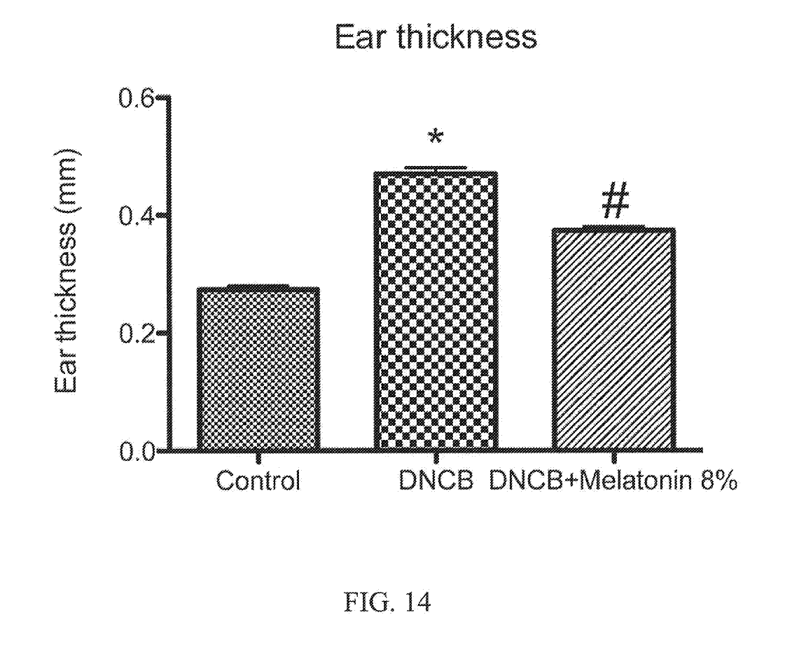

The results were shown in FIGS. 13 and 14. In the DNCB-stimulated group (representing AD), severe skin erythema, skin excoriations, severe skin thickening and scaling, and poor hair growth were noted (clinical score=10) compared with Control group (clinical score=0). In DNCB-stimulated mice treated with 0.1% melatonin, dermatitis mildly improved, with less skin excoriation, and skin thickening and scaling were milder (clinical score=7). In DNCB-stimulated mice treated with 8% melatonin, dermatitis improved further, with no skin excoriation or scaling, and skin thickening was improved. Hair growth was also improved (clinical score=2). From the gross findings, a dose-response relationship with melatonin treatment was observed (FIG. 13). Ear thickness was also significantly improved after 8% melatonin treatment (FIG. 14). These results showed that 0.1%, 1%, and 8% topical melatonin treatment improved the gross skin findings in a dose-dependent manner, whereby 8% topical melatonin improved the skin severity most.

### 4. Serum IgE Measurement

The serum IgE levels were measured on the last day of the experiment (Day 15) in the same manner as those described in Example 5.

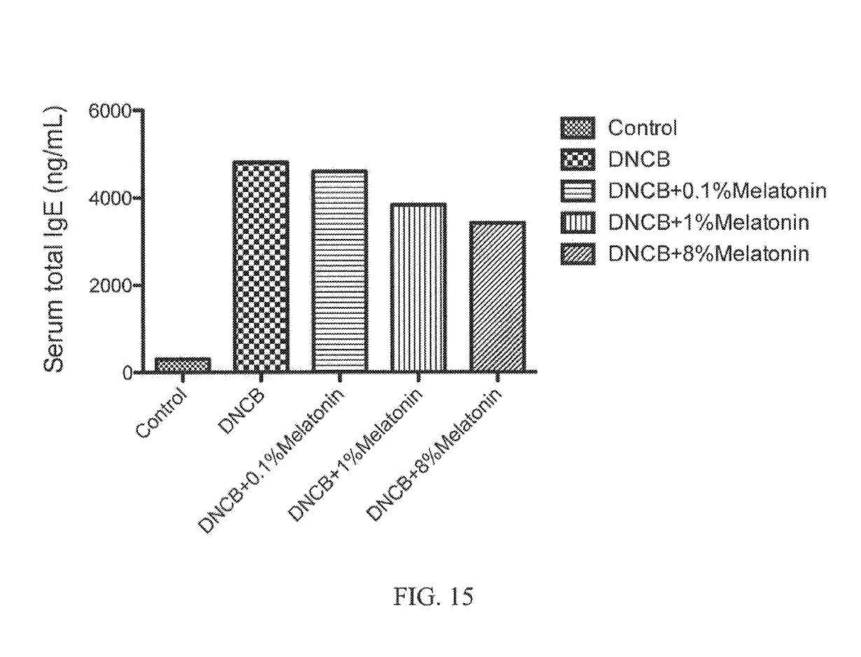

Referring to FIG. 15, the serum total IgE levels in DNCB-stimulated mice were reduced after topical melatonin treatment, and there was dose-response relationship, whereby 8% topical melatonin treatment lowered the total serum IgE most in FIG. 15.

### 5. Statistical Analysis

Average data were presented as mean±SD. It was performed statistical analysis using IBM SPSS for MAC, version 20. Comparisons between different experimental groups were conducted using ANOVA followed by Bonferroni test. Statistical difference was considered significant when p<0.05.

The exemplary embodiments of the present disclosure demonstrate that topical administration of melatonin improves the gross skin findings and pathology scoring in an atopic dermatitis model. Melatonin treatment also decreases both mast cell and T cell infiltration in the skin, decreases the total serum IgE level, and decreases skin expression of the inflammatory cytokines IL-4 and IL-17. Different concentrations of melatonin (e.g., an amount of from 0.001% to 30% by weight, based on the total weight of the composition) exhibit the great effect on treatment of atopic dermatitis, and such effect is dose-dependent. These results indicate that the melatonin associated compound significantly improves the severity of dermatitis and thus is useful for treating AD.

The present general inventive concept has been described using exemplary preferred embodiments. However, it is to be understood that the scope of the present disclosure is not limited to the disclosed embodiments. On the contrary, it is intended to cover various modifications and similar rearrangement. The scope of the claims therefore should be accorded the broadest interpretation so as to encompass all such modifications and similar arrangements.

The references listed below cited in the application are each incorporated by reference as if they were incorporated individually.
1. Bieber T. Atopic dermatitis. N Engl J Med. 2008; 358(14):1483-1494.
2. Camfferman D, Kennedy J D, Gold M, Martin A J, Lushington K. Eczema and sleep and its relationship to daytime functioning in children. Sleep Med Rev. 2010; 14(6):359-369.
3. Ricci G, Bendandi B, Bellini F, Patrizi A, Masi M. Atopic dermatitis: quality of life of young Italian children and their families and correlation with severity score. Pediatr Allergy Immunol. 2007; 18(3): 245-249.
4. Lewis-Jones S. Quality of life and childhood atopic dermatitis: themisery of living with childhood eczema. Int J Clin Pract. 2006; 60(8): 984-992.
5. Hon K L, Lam M C, Leung T F, Chow C M, Wong E, Leung A K. Assessing itch in children with atopic dermatitis treated with tacrolimus: objective versus subjective assessment. Adv Ther. 2007; 24(1):23-28.
6. Leo H L, Bender B G, Leung S B, Tran Z V, Leung D Y. Effect of pimecrolimus cream 1% on skin condition and sleep disturbance in children with atopic dermatitis. J Allergy Clin Immunol. 2004; 114 (3):691-693.
7. Kelsay K. Management of sleep disturbance associated with atopic dermatitis. J Allergy Clin Immunol. 2006; 118(1): 198-201.
8. Richardson G S, Roehrs T A, Rosenthal L, Koshorek G, Roth T. Tolerance to daytime sedative effects of H1 antihistamines. J Clin Psychopharmacol. 2002; 22(5):511-515.
9. Levander S, Ståhle-Bäckdahl M, Higermark O. Peripheral antihistamine and central sedative effects of single and continuous oral doses of cetirizine and hydroxyzine. Eur J Clin Pharmacol. 1991; 41(5):435-439.
10. Choi Y Y, Kim M H, Kim J H, Jung H S, Sohn Y, Choi Y J, Hwang M K, Kim S H, Kim J, Yang W M. Schizonepeta tenuifolia inhibits the development of atopic dermatitis in mice. Phytother Res. 2013; 27(8):1131-5.
11. Kim T H, Jung J A, Kim G D, Jang A H, Ahn H J, Park Y S, Park C S. Melatonin inhibits the development of 2,4-dinitrofluorobenzene-induced atopic dermatitis-like skin lesions in N C/Nga mice. J Pineal Res. 2009; 47(4):324-9.
12. Yoon H J, Jang M S, Kim H W, Song D U, Nam K I, Bae C S, Kim S J, Lee S R, Ku C S, Jang D I, Ahn B W. Protective effect of diet supplemented with rice prolamin extract against DNCB-induced atopic dermatitis in BALB/c mice. BMC Complement Altern Med. 2015; 15:353.

What is claimed is:

1. A method for treating atopic dermatitis in a subject in need thereof comprising administering to the subject a composition comprising a therapeutically effective amount of a melatonin associated compound and a pharmaceutically acceptable excipient.

2. The method according to claim 1, wherein the melatonin associated compound is at least one selected from the group consisting of melatonin (MLT), melatonin receptor agonists, Ramelton, Agomelatine, Tasimelteon, TIK-301, halogenated melatonin, 2-iodomelatonin, 2-bromomelatonin, 2-phenylmelatonin, 6-chloromelatonin, and the following compounds represented by Formulae 1-28: 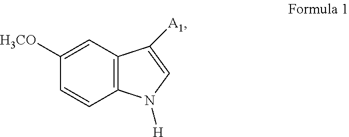
wherein A₁ is CH₂CH₂NHCOCH₃; 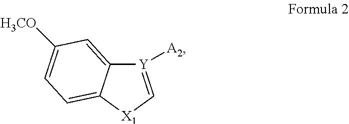
wherein A₂ is CH₂CH₂NHCOCH₃, and X₁ is S, O or CH₂ when Y is CH, or X₁ is NH when Y is N; 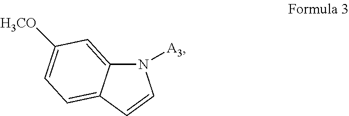
wherein A₃ is CH₂CH₂NHCOCH₃; 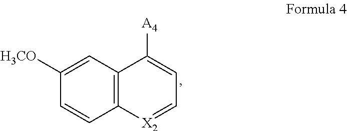
wherein A₄ is CH₂CH₂NHCOCH₃, and X₂ is CH or N; 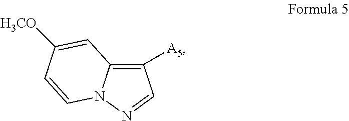
wherein A₅ is CH₂CH₂NHCOCH₃; 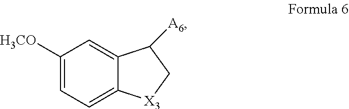
wherein A₆ is CH₂CH₂NHCOCH₃, and X₃ is NH or CH₂; 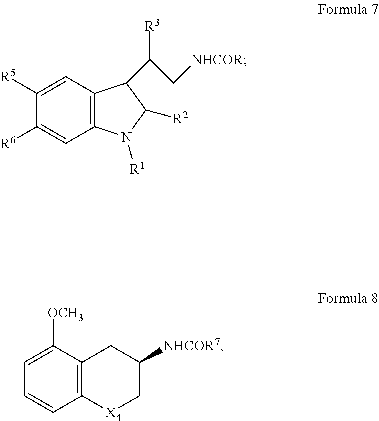
wherein X₄ is CH₂ when R⁷ is methyl, or X₄ is O when R⁷ is n-propyl; 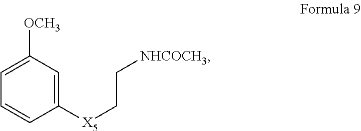
wherein X₅ is CH₂ or NCH₃; 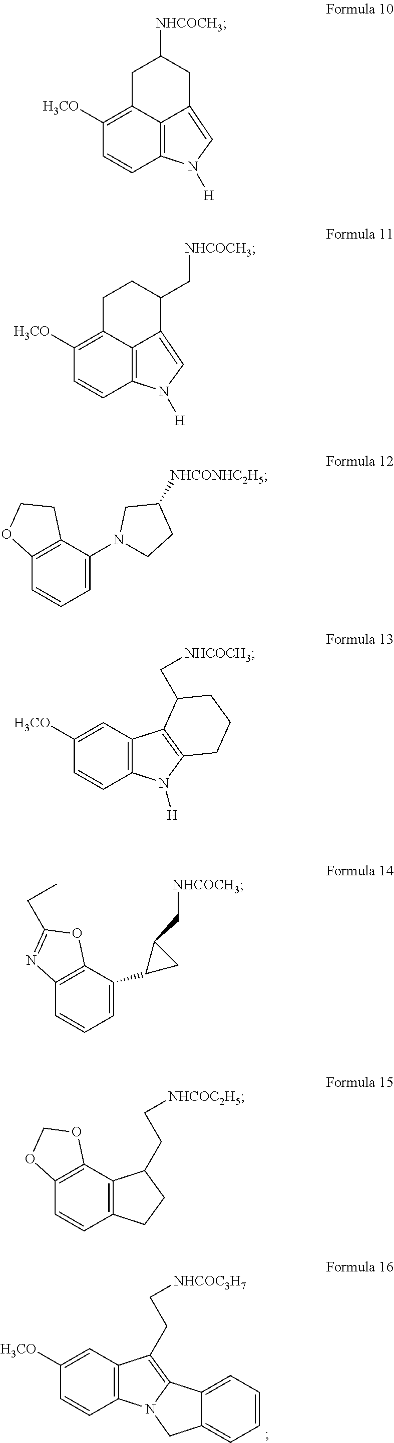 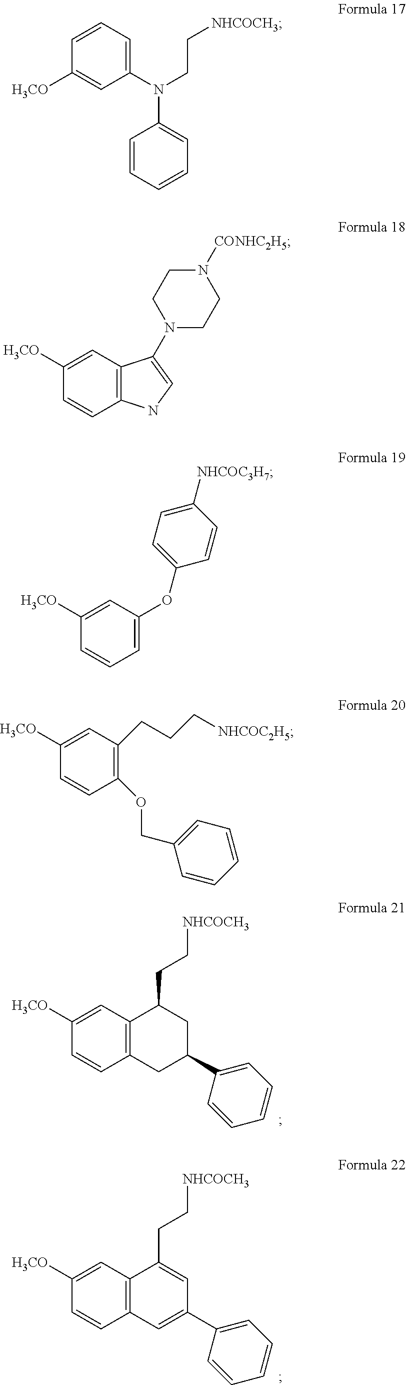 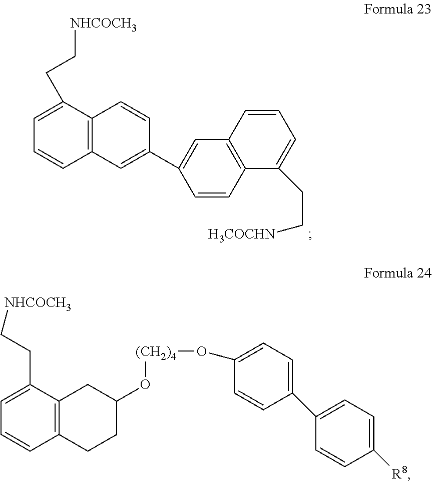
wherein R⁸ is COOH or CH₂OH; 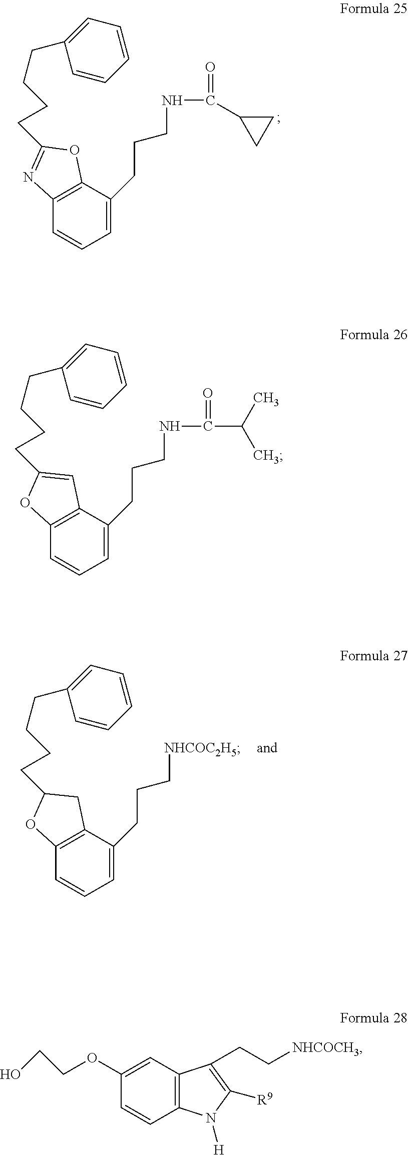
wherein R⁹ is H or Br.

3. The method according to claim 1, wherein the melatonin associated compound in the composition is in an amount of from 0.001% to 30% by weight.

4. The method according to claim 3, wherein the melatonin associated compound in the composition is in an amount of from 0.1% to 10% by weight.

5. The method according to claim 4, wherein the melatonin associated compound in the composition is in an amount of from 3% to 8% by weight.

6. The method according to claim 1, wherein the melatonin associated compound is administered to the subject in an amount ranging from 0.0001 mg/day to 50 mg/day.

7. The method according to claim 6, wherein the melatonin associated compound is administered to the subject in an amount ranging from 0.01 mg/day to 25 mg/day.

8. The method according to claim 7, wherein the melatonin associated compound is administered to the subject in an amount of about 3 mg/day.

9. The method according to claim 1, wherein the composition is administered to the subject 1 to 4 times daily.

10. The method according to claim 9, wherein the composition is administered to the subject 2 times daily.

11. The method according to claim 1, wherein the composition is orally, intravenously, intraperitoneally, intramuscularly, externally or transdermally administered to the subject.

12. The method according to claim 1, wherein the composition is administered to the subject in a period ranging from 3 days to 2 years.

13. The method according to claim 12, wherein the composition is administered to the subject in a period ranging from 2 weeks to 12 months.

14. The method according to claim 13, wherein the composition is administered to the subject in a period ranging from 2 to 4 weeks.

15. The method according to claim 1, further comprising administering an additional therapeutic agent for treating atopic dermatitis at the same time of administering the melatonin associated compound, wherein the additional therapeutic agent is at least one selected from a steroid, an anti-histamine agent, cetirizine, loratadine, chlorpheniramine, and a natural or synthetic immune inhibitor.

16. The method according to claim 1, further comprising administering an additional therapeutic agent for treating atopic dermatitis separately from administering the melatonin associated compound, wherein the additional therapeutic agent is at least one selected from a steroid, an anti-histamine agent, cetirizine, loratadine, chlorpheniramine, and a natural or synthetic immune inhibitor.

17. The method according to claim 1, wherein the subject is mammal.

18. The method according to claim 17, wherein the subject is human.

19. The method according to claim 1, wherein the age of the subject is in a range of from 0 to 18 years old.

20. The method according to claim 1, being in combination with an additional therapy for preventing or treating atopic dermatitis, wherein the additional therapy is at least one chosen from phototherapy, skin care, protection from allergen exposure, or administration of a skin moisturizer, an adjuvant, an antioxidant, a vitamin, or a mineral.
